(19) United States
(12) Patent Application Publication
(10) Pub. No.: US 2019/0382838 A1
(43) Pub. Date: Dec. 19, 2019

(54) Methods For Single-Molecule Fluorescence Amplification Of RNA

(71) Applicant: THE TRUSTEES OF THE UNIVERSITY OF PENNSYLVANIA, Philadelphia, PA (US)

(72) Inventors: Arjun RAJ, Wynnewood, PA (US); Sara H. ROUHANIFARD, Camden, NJ (US)

(21) Appl. No.: 16/428,622

(22) Filed: May 31, 2019

Related U.S. Application Data
(60) Provisional application No. 62/679,086, filed on Jun. 1, 2018.

(51) Int. Cl. C12Q 1/6876; G01N 1/30

(52) U.S. Cl. CPC C12Q 1/6876; G01N 2001/305; G01N 1/30

(57) ABSTRACT

The present invention provides novel methods for exponential amplification of labeled nucleic acids with high sensitivity and specificity. In one aspect, the invention includes a method for exponentially amplifying the signal of a fluorescently labeled primary click-amplifying FISH (clampFISH) probe. In another aspect, the invention includes a method for labeling a target nucleic acid in a sample. In yet another aspect, the invention includes a method for detecting a fluorescently labeled target nucleic acid in a sample. The present invention also provides a kit for use with the methods of the invention.

### CROSS-REFERENCE TO RELATED APPLICATION

This application claims priority under 35 U.S.C. § 119(e) to U.S. Provisional Patent Application Ser. No. 62/679,086 filed Jun. 1, 2018. The entire content of this application is hereby incorporated by reference herein.

### STATEMENT REGARDING FEDERALLY SPONSORED RESEARCH OR DEVELOPMENT

This invention was made with government support under Grant Nos. HL129998, HG007743, GM120929 and EB019767 awarded by the National Institutes of Health (NIH) and under Grant No. 1350601 awarded by the National Science Foundation (NSF). The government has certain rights in the invention.

### BACKGROUND OF THE INVENTION

RNA fluorescence in situ hybridization (RNA FISH) techniques have evolved into powerful tools for directly measuring RNA levels. In particular, advances in oligonucleotide technologies and microscope sensitivity have enabled researchers to detect individual RNA molecules directly inside single cells with high efficiency (Femino et al., Science 280, 585-590 (1998); Raj et al., Nat. Methods 5, 877-879 (2008)). Yet, despite the power of direct-labeling approaches, one persistent issue has been that the total signal generated by probes directly bound to the target is necessarily relatively low, and thus detection requires high-power microscopy. This prevents its application in several other contexts, such as low-power microscopy or flow cytometry, where signals must be considerably higher for reliable analysis.

There are currently a number of different signal amplification techniques available for combination with RNA FISH, but each suffers from particular limitations. One class of scheme relies on targeting an enzyme to the RNA of interest that catalyzes the production of fluorescence. These enzymes can either directly generate localized fluorescence (e.g., tyramide signal amplification (Chen et al., Anal. Chem. 83, 7269-7275 (2011)), Perkin-Elmer; or enzyme ligated fluorescence, Thermo/Invitrogen (Lu & Tsourkas, Nucleic Acids Res. 37, e100 (2009)), or can catalyze a “rolling circle” nucleic acid amplification (Baner et al., Nucleic Acids Res. 26, 5073-5078 (1998); Larsson et al., Nat. Methods 7, 395-397 (2010); Monsur et al., Chem. Soc. Rev. 43, 3324-3341 (2014)). These methods lead to large gains in fluorescence due to the rapid and continuous activity of the enzyme, but suffer in sensitivity because of the difficulties in getting large, bulky enzymes through the fixed cellular environment to the target molecule. Given that many important mRNA are present at very low abundances, often 10 or fewer per cell, sensitivities of around 10% (typical for these schemes (Larsson et al. Nat. Methods 1, 227-232 (2004); Larsson et al., Nat. Methods 7, 395-397 (2010)) could lead to significant false negatives.

Meanwhile, there are a number of non-enzymatic amplification methods, most notably the hybridization chain reaction (Dirks & Pierce, Proc. Natl. Acad. Sci. U.S.A. 101, 15275-15278 (2004); Choi et al., ACS Nano 8, 4284-4294 (2014); Shah et al. Development dev.138560 (2016)) and branched DNA methods (Lau et al., Lancet 341, 1501-1504 (1993); Kern al., 34, 3196-3202 (1996); Battich et al., Nat. Methods 10, 1127-1133 (2013)). These methods rely on hybridization to amplify signal by creating larger DNA scaffolds to which fluorescent probes can attach. However, these techniques generally suffer from two problems: 1. background probe binding leads to a high number of false positive signals, because each false binding event is amplified to be indistinguishable from true binding, and 2. the degree of amplification is typically rather limited, thus not boosting signals of lowly expressed genes to the point where they are sufficient for flow cytometry or low-power microscopy. For HCR, amplification is fundamentally limited by the fact that the amplification scheme is linear, and so when a particular chain terminates, there can be no further amplification. Branched DNA assays have a limit in terms of amplification because each step of branching requires a new set of oligonucleotides, so a fixed pool of oligonucleotides will have at most a certain amount of amplification. Indeed, in the case of the branched DNA assays, the most quantitative measurements to date measured only a 2-3 fold gain in signal to noise despite an amplification of 100-fold (Battich et al., Nat. Methods 10, 1127-1133 (2013)), while hybridization chain reaction yields a similar degree of amplification (Choi et al., ACS Nano 8, 4284-4294 (2014)).

Thus, there is a need in the art for compositions and methods for exponential amplification of RNA FISH signal with high sensitivity and specificity. The present invention addresses this unmet need.

### SUMMARY OF THE INVENTION

One aspect of the invention provides a method for exponentially amplifying the signal of a fluorescently labeled primary click-amplifying FISH (clampFISH) probe, the method comprising: hybridizing the primary clampFISH probe to a target nucleic acid in a sample to form a primary sample; adding a click chemistry agent that covalently locks the primary clampFISH probe to the target nucleic acid in the primary sample; contacting the primary sample with a set of secondary clampFISH probes that bind to the primary clampFISH probe and adding a click chemistry agent that covalently locks the set of secondary clampFISH probes to the primary clampFISH probe to form a secondary sample; contacting the secondary sample with a set of tertiary clampFISH probes that bind to each secondary clampFISH probe and adding a click chemistry agent that covalently locks the set tertiary clampFISH probes to each secondary clampFISH probe to form a tertiary sample; contacting the tertiary sample with a set of secondary clampFISH probes that bind to each tertiary clampFISH probe and adding a click chemistry agent that covalently locks the secondary clampFISH probes to each tertiary clampFISH probe; and, repeating steps (d) and (e) until a desired level of fluorescent signal is achieved thereby exponentially amplifying the level of fluorescent signal of the primary clampFISH probe.

In another aspect, the invention provides a method for labeling a target nucleic acid in a sample, the method comprising: contacting the sample with a fixative, thereby producing a fixed sample; contacting the fixed sample with a hybridization solution, the hybridization solution comprising one or more primary click-amplifying FISH (clampFISH) probes which hybridize to one or more regions of the target nucleic acid and adding a click chemistry agent that covalently locks the one or more primary clampFISH probes to the one or more regions of the target nucleic acid to form a primary sample; contacting the primary sample with a set of secondary clampFISH probes that bind to the one or more primary clampFISH probes and adding a click chemistry agent that covalently locks the set of secondary clampFISH probes to the one or more primary clampFISH probes to form a secondary sample; contacting the secondary sample with a set of tertiary clampFISH probes that bind to each secondary clampFISH probe and adding a click chemistry agent that covalently locks the set tertiary clampFISH probes to each secondary clampFISH probe to form a tertiary sample; contacting the tertiary sample with a set of secondary clampFISH probes that bind to each tertiary clampFISH probes and adding a click chemistry agent that covalently locks the set secondary clampFISH probes to each tertiary clampFISH probe; and, repeating steps (d) and (e) until a desired level of labeling of the target nucleic acid is achieved.

In another aspect, the invention provides a method for detecting a fluorescently labeled target nucleic acid in a sample, the method comprising: contacting the sample with a fixative, thereby producing a fixed sample; contacting the fixed sample with a hybridization solution, the hybridization solution comprising one or more a primary click-amplifying FISH (clampFISH) probes which hybridize to one or more regions of the target nucleic acid and adding a click chemistry agent that covalently locks the one or more primary clampFISH probes to the one or more regions of the target nucleic acid to form a primary sample; contacting the primary sample with a set of secondary clampFISH probes that bind to the one or more primary clampFISH probes and adding a click chemistry agent that covalently locks the set of secondary clampFISH probes to the one or more primary clampFISH probes to form a secondary sample; contacting the secondary sample with a set of tertiary clampFISH probes that bind to each secondary clampFISH probe and adding a click chemistry agent that covalently locks the set tertiary clampFISH probes to each secondary clampFISH probe to form a tertiary sample; contacting the tertiary sample with a set of secondary clampFISH probes that bind to each tertiary clampFISH probes and adding a click chemistry agent that covalently locks the set secondary clampFISH probes to each tertiary clampFISH probe; and, repeating steps (d) and (e) until a desired level of fluorescent signal of the labeled target nucleic acid is achieved; and detecting the fluorescent signal of the labeled target nucleic acid wherein the level of fluorescent signal is exponentially amplified.

In various embodiments, step (f) is repeated 1, 2, 3, 4, 5, 6, 7, 8, 9 or 10 times.

In various embodiments, the set of secondary and tertiary clampFISH probes comprises at least 2 probes.

In various embodiments, each probe of the set of secondary and tertiary clampFISH probes binds to a different region of their respective nucleic acid target.

In various embodiments, the length of the primary, secondary and tertiary clampFISH probes is about 150 nucleic acids.

In various embodiments, the primary, secondary and tertiary clampFISH probes are complementary through their 3′ and 5′ ends to two regions of their respective nucleic acid target.

In various embodiments, the 3′ and 5′ ends of each primary, secondary and tertiary clampFISH probe comprise a binding arm and an adapter of about 15 and 10 nucleic acids respectively.

In various embodiments, the 3′ and 5′ ends of each primary, secondary and tertiary clampFISH probe comprise an azide and an alkyne group respectively or an alkyne and an azide group respectively.

In various embodiments, the internal region of each primary, secondary and tertiary clampFISH probe comprises at two separate locations an alkyne and an azide group.

In various embodiments, the click chemistry agent catalyzes an azide-alkyne cycloaddition thereby covalently locking the primary, secondary and tertiary clampFISH probes around the their respective nucleic acid target.

In various embodiments, the click chemistry is catalyzed by a copper(I), a copper(II) or a ruthenium.

In various embodiments, the primary, secondary and tertiary clampFISH probes are labeled by fluorophore.

In various embodiments, the signal of the labeled primary, secondary and tertiary clampFISH probes is detected by a fluorescent in situ hybridization (FISH).

In various embodiments, the primary, secondary and tertiary clampFISH probes is a DNA.

In various embodiments, the target nucleic acid is a RNA.

In various embodiments, the RNA is selected from the group consisting of messenger RNA, intronic RNA, exonic RNA, and non-coding RNA.

In various embodiments, the target nucleic acid comprises a splice junction. In various embodiments, the method is used to identify a splice junction in the target nucleic acid.

In various embodiments, the fluorescent signal is amplified more than 10, more than 50, more than 100 and more than 500 folds as compared to a control labeled with a standard FISH or labeled with a primary, secondary and tertiary clampFISH probes but without repeating the step (f).

In various embodiments, the detection of target nucleic acid in a sample is achieved by using at least one method selected from the group consisting of: low-magnification microscopy and flow cytometry.

In various embodiments, the target nucleic acid in a sample is further processed for expansion microscopy.

In another aspect, the invention provides a kit comprising at set of primary, secondary and tertiary click-amplifying FISH (clampFISH) probes and a click chemistry agent for signal amplification and detection of nucleic acids in a sample and instructions for use thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1E are series of images and graphs illustrating the design and validation of clampFISH technology. FIG. 1A: Molecular inversion probes (MIP) (padlock probes) topologically wrap around target (left) and can be ligated to connect the 5′ and 3′ ends of the probe (right). FIG. 1B: Click chemistry (CuAAC) may be used to ligate the ends together non-enzymatically. FIG. 1C: Workflow for clampFISH: multiple, primary clampFISH probes bind to the target of interest. Secondary clampFISH probes bind 2:1 to each primary clampFISH probe, and tertiary clampFISH probes bind 2:1 to each secondary clampFISH probe. In a subsequent round, the secondary probes again bind 2:1 to the tertiary probes and so on, thus providing exponential amplification. FIG. 1D: Single cell tracking of GFP clampFISH signal on WM983b-GFP cells across 10 rounds of amplification in the presence of click ligation (top) compared to GFP clampFISH signal in the absence of click ligation (middle). Single cell tracking of the same cell line without GFP expression across rounds to assess background signal (below). FIG. 1E: Mean fluorescence intensity of GFP clampFISH signal on WM983b-GFP cells across 10 rounds of clampFISH.

FIGS. 2A-2C are series of graphs and images depicting applications of the clampFISH amplification of RNA. FIG. 2A: ClampFISH was applied to a mixed population of WM983b cells with and without GFP expression and analyzed the separation by flow cytometry across 10 rounds of amplification. Cells were gated on GFP expression and are displayed in green. FIG. 2B: 20% WM983b cells stably expressing GFP were mixed with 80% WM983b cells and probed for GFP mRNA using clampFISH probes (top). Imaging was performed using 10×, 20×, 60× and 100× magnification objectives in the same positions and compared to single molecule fluorescent in situ hybridization (smFISH) of GFP mRNA using the same fluorophore (bottom). FIG. 2C: Fluorescence image of clampFISH targeting GFP in cultured WM983b-GFP cells (left) and Neat1 lncRNA in cultured WM983b cells (right).

FIG. 3 is a series of images demonstrating that clampFISH technology can be used on methanol fixed cells as well as formaldehyde fixed cells. The images illustrate GFP clampFISH probes that bind to GFP mRNA target in methanol fixed cells and formaldehyde fixed cells. Arrows indicate clampFISH signal colocalized with GFP smFISH using both fixation methods.

FIGS. 4A-4B are series of images demonstrating that clampFISH probes can be used as a proximity ligation assay. FIG. 4A: Drawings illustrating different scenarios where (i) the two arms of the primary clampFISH probe are bound to the target and click chemistry occurs or not (first and second drawing from the left); (ii) where a mismatch occurs and only one arm of primary clampFISH probe binds to target (third drawing from the left); and (iii) where in the absence of a target mRNA, no binding of the primary clampFISH occurs (fourth image from left). FIG. 4B: WM983b cells stably expressing a GFP tag are probed with GFP clampFISH probes (top row) and assessed for survival after stringency washes (bottom row). First set of images from the left, most of the spots remain after stringent washes when the two binding arms of a primary clampFISH probe come together in the presence of a click reaction. Second set of images from the left, without click reaction, the spots look the same, however they do not survive stringent washes. Third set of images from the left, with a one arm mismatch and when only one of the arms of the primary clampFISH probe binds specifically, some binding of the probe occurs on the target RNA, but it washes away during stringent washes, even in the presence of the reagents needed for a click reaction. Forth set of images from the left, control with the clampFISH probes, the reagents needed for a click reaction but without any target RNA.

FIG. 5 is a series of images illustrating multiplexed clampFISH. NEAT1 clampFISH probes are hybridized to NEAT1 mRNA using a unique backbone sequence. Probes bind specifically to NEAT1 mRNA that localizes to nuclear paraspeckles (arrows). For NEAT1 mRNA, the signal is detected using an alexa-594 labeled smFISH probe that binds to the terminal, unlabeled clampFISH probe. For the GFP mRNA clampFISH probe, a Cy5 fluorophore is built into the backbone sequence.

FIGS. 6A-6D are series of images illustrating the Generation of ClampFISH probes. (FIG. 6A) Diagram of individual pieces of each clampFISH probe. (FIG. 6B) Linear diagram of clampFISH probe ligations scheme. (FIG. 6C) ClampFISH probe ligation protocol. (FIG. 6D) 15% TBE-UREA gel showing separation of individual pieces, ligation product, and product after purification.

FIGS. 7A-7B are series of images depicting the colocalization of GFP mRNA signal with GFP protein. (FIG. 7A) ClampFISH images as shown in FIGS. 2A-2C with a panel showing which cells are GFP positive (green). (FIG. 7B) smFISH images as shown in FIGS. 2A-2C with a panel showing which cells are GFP positive (green).

FIGS. 8A-8C are series of images and graphs demonstrating some of the applications of clampFISH amplification of RNA (images are contrasted independently). (FIG. 8A) (center) 20× image of fixed-frozen 5 uM mouse kidney section stained with round 4 clampFISH probes targeting PODXL. (left) 60× image of mouth endothelium by round 4 clampFISH and by smFISH. (right) 60× image of podocyte by round 4 clampFISH and by smFISH (representative images shown of 2 biological replicates). (FIG. 8B) ClampFISH was applied to a mixed population of MDA-MB 231 cells with and without GFP expression and analyzed the separation by flow cytometry across 8 rounds of amplification. Cells were gated on GFP expression and are displayed in green. (FIG. 8C) (top) Fluorescent micrographs of round 6 clampFISH targeting GFP mRNA and Neatl lncRNA in cultured WM983b-GFP cells (bottom) fluorescent micrographs of smFISH targeting GFP mRNA and Neat1 lncRNA in cultured WM983b-GFP cells using the same dye (images representative of 2 biological replicates; scale bars are 20 um for the images, and 5 um for the inlay).

FIG. 9 is the enlarged center panel from FIG. 8A showing nonspecific binding of clampFISH probes on mouse kidney tissues.

FIG. 10 is a series of flow cytometry with various addition of blocking reagents to the hybridization buffer. MDA-MB 231 cells expressing GFP were mixed with MDA-MB 231 cells not expressing GFP at 50%. The mixed cell population was subsequently stained with clampFISH probes targeting GFP mRNA and the separation was assessed with the addition of different blocking reagents in the hybridization buffer.

FIGS. 11A-11B are series of a diagram and box plots depicting clampFISH expansion. (FIG. 11A) Expansion clampFISH workflow. (FIG. 11B) Expansion clampFISH samples were assessed for spot intensity and mRNA counts per cell with smFISH and clampFISH on WM983bGFP cells.

FIGS. 12A-12B are series of diagrams and images showing multiplexed 3 RNA targets on HeLa cells. (FIG. 12A) Schematic diagram of probe hybridization scheme. Unique clampFISH probe sets are designed for each target, and probed at the final round with a smFISH probe labeled with a unique fluorophore (represented with ⋆). (FIG. 12B) Fluorescent micrographs of individual probe channels: (from left) NEAT1 lncRNA labeled with ATTO700, HIST1H4E mRNA labeled with ATTO 488, and LMNA mRNA labeled with Alexa 594 and an overlay on the far right. (top) 100× magnification with 20 um scale bars, (bottom) 20× magnification with 20 um scale bars.

FIG. 13 is a series of images showing multiplexed ClampFISH to Round 7 bleedthrough. Cells were stained with clampFISH probes to round 7 individually and assessed using the same exposure times from the multiplexing experiment in FIGS. 12A-12B for bleedthrough.

FIG. 14 is a series of diagrams and images demonstrating that ClampFISH probes require adjacent hybridization of probe arms and click reaction to survive harsh conditions. 20 μm scale bar.

FIGS. 15A-15C are series of tables listing examples of clampFISH probes (SEQ ID NOs: 1-148). (FIG. 15A) ClampFISH sequences for NEAT1 (SEQ ID NOs: 1-61). (FIG. 15B) ClampFISH sequences for HIST1H4E (SEQ ID NOs: 62-101 and 148). (FIG. 15C) ClampFISH sequences for LMNA (SEQ ID NOs: 102-147).

### DETAILED DESCRIPTION

The present invention provides novel methods for exponential amplification of RNA FISH signal with high sensitivity and specificity. The present method thereby allows for FISH to be used in high-throughput screening methods and diagnostics.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±20% or ±10%, ±5%, ±1%, or ±0.1% from the specified value, as such variations are appropriate.

As used herein, the terms “alkyne group”, “alkyne moiety”, “alkyne” or “alkynyl” are used herein interchangeably. These terms employed alone or in combination with other terms, mean, unless otherwise stated, a stable straight, branched, or cyclic chain hydrocarbon group with a triple carbon-carbon bond, having the stated number of carbon atoms. Non-limiting examples include ethynyl and propynyl, and the higher homologs and isomers. Exemplary alkyl groups of use in the present invention contain between about one and about twenty five carbon atoms (e.g. methyl, ethyl and the like). Straight, branched or cyclic hydrocarbon chains having eight or fewer carbon atoms will also be referred to herein as “lower alkyl” (e.g. cyclooctyne). In addition, the term “alkyl” as used herein further includes one or more substitutions at one or more carbon atoms of the hydrocarbon chain fragment.

The term “click chemistry,” as used herein, refers to the Huisgen cycloaddition or the 2,3-dipolar cycloaddition between an azide and a terminal alkyne to form a 1,2,4-triazole. Such chemical reactions can use, but are not limited to, simple heteroatomic organic reactants and are reliable, selective, stereospecific, and exothermic. As used herein, click chemistry also refers to a strain promoted azide alkyne cycloaddition (SpAAC) where a cyclooctyne is able to undergo azide-alkyne Huisgen cycloaddition under mild, physiological conditions in the absence of a copper(I) catalyst.

The term “mutation” as used herein refers to any change of one or more nucleotides in a nucleotide sequence.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 75% homology.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene. A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

As used herein, the term “covalently locks” refers to the interaction formed between clampFISH probes and the one or more regions of the target nucleic acid or between the various clampFISH probes, in each case as shown in the figures. Covalent locking does not require a covalent bond between the molecules.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids, which have been substantially purified from other components, which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

The term “fluorophore” as used herein refers to a composition that is inherently fluorescent or demonstrates a change in fluorescence upon binding to a biological compound or metal ion, i.e., fluorogenic. Fluorophores may contain substituents that alter the solubility, spectral properties or physical properties of the fluorophore. Numerous fluorophores are known to those skilled in the art and include, but are not limited to coumarin, cyanine, benzofuran, a quinoline, a quinazolinone, an indole, a benzazole, a borapolyazaindacene and xanthenes including fluorescein, rhodamine and rhodol as well as other fluorophores known in the art.

A “portion” of a polynucleotide means at least at least about five to about fifty sequential nucleotide residues of the polynucleotide. It is understood that a portion of a polynucleotide may include every nucleotide residue of the polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label,” as used herein, refers to a chemical moiety or protein that is directly or indirectly detectable (e.g. due to its spectral properties, conformation or activity) when attached to a target or compound and used in the present methods, including reporter molecules and carrier molecules. The label can be directly detectable (fluorophore) or indirectly detectable (hapten or enzyme). Such labels include, but are not limited to, radiolabels that can be measured with radiation-counting devices; pigments, dyes or other chromogens that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems, for example. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a calorimetric substrate (e.g., tetramethylbenzidine (TMB)) or a fluorogenic substrate such as Amplex Red reagent (Molecular Probes, Inc.) to detect the presence of HRP. Numerous labels are known by those of skill in the art and include, but are not limited to, particles, fluorophores, haptens, enzymes and their calorimetric, fluorogenic and chemiluminescent substrates and other labels known in the art.

“Naturally occurring” as used herein describes a composition that can be found in nature as distinct from being artificially produced. For example, a nucleotide sequence present in an organism, which can be isolated from a source in nature and which has not been intentionally modified by a person in the laboratory, is naturally occurring.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The terms “patient,” “subject,” “individual,” and the like are used interchangeably herein, and refer to any animal, or cells thereof whether in vitro or in situ, amenable to the methods described herein. Preferably, the patient, subject or individual is a mammal, and more preferable, a human.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to new methods for exponential amplification of nucleic acid using a click-amplifying FISH (ClampFISH) probes. The present invention addresses the unmet need for with high sensitivity and specificity FISH together with a high-throughput screening method.

### Methods of the Invention

In situ hybridization (ISH) is a method well known in the art that utilizes nucleic acid probes to detect DNA or RNA targets in cells via Watson-Crick base pairing of the probe to the target.

The present invention relates to methods for labeling, amplifying the labeling and reliably detecting one or more target nucleic acids in a sample using a click-amplifying FISH (clampFISH). The present invention may be utilized in any FISH application known in the art. For example, the present invention may be used in methods to detect the presence of a target sequence, the location of a target sequence, the presence of a mutation, the presence of a splice junction and the like. The methods of the invention can be generally described as follow.

In one aspect, the invention includes a method for exponentially amplifying the signal of a fluorescently labeled primary click-amplifying FISH (clampFISH) probe. The method of the invention comprises the following steps: (a) hybridizing the primary clampFISH probe to a target nucleic acid in a sample to form a primary sample; (b) adding a click chemistry agent that covalently locks the primary clampFISH probe to the target nucleic acid in the primary sample; (c) contacting the primary sample with a set of secondary clampFISH probes that bind to the primary clampFISH probe and adding a click chemistry agent that covalently locks the set of secondary clampFISH probes to the primary clampFISH probe to form a secondary sample; (d) contacting the secondary sample with a set of tertiary clampFISH probes that bind to each secondary clampFISH probe and adding a click chemistry agent that covalently locks the set tertiary clampFISH probes to each secondary clampFISH probe to form a tertiary sample; (e) contacting the tertiary sample with a set of secondary clampFISH probes that bind to each tertiary clampFISH probe and adding a click chemistry agent that covalently locks the secondary clampFISH probes to each tertiary clampFISH probe; and, (f) repeating steps (d) and (e) until a desired level of fluorescent signal is achieved thereby exponentially amplifying the level of fluorescent signal of the primary clampFISH probe.

In another aspect, the invention includes a method for labeling a target nucleic acid in a sample. The method of the invention comprises the following steps: (a) contacting the sample with a fixative, thereby producing a fixed sample; (b) contacting the fixed sample with a hybridization solution, the hybridization solution comprising one or more primary click-amplifying FISH (clampFISH) probes which hybridize to one or more regions of the target nucleic acid and adding a click chemistry agent that covalently locks the one or more primary clampFISH probes to the one or more regions of the target nucleic acid to form a primary sample; (c) contacting the primary sample with a set of secondary clampFISH probes that bind to the one or more primary clampFISH probes and adding a click chemistry agent that covalently locks the set of secondary clampFISH probes to the one or more primary clampFISH probes to form a secondary sample ; (d) contacting the secondary sample with a set of tertiary clampFISH probes that bind to each secondary clampFISH probe and adding a click chemistry agent that covalently locks the set tertiary clampFISH probes to each secondary clampFISH probe to form a tertiary sample; (e) contacting the tertiary sample with a set of secondary clampFISH probes that bind to each tertiary clampFISH probes and adding a click chemistry agent that covalently locks the set secondary clampFISH probes to each tertiary clampFISH probe; and, (f) repeating steps (d) and (e) until a desired level of labeling of the target nucleic acid is achieved.

In yet another aspect, the invention includes a method for detecting a fluorescently labeled target nucleic acid in a sample. The method of the invention comprises the following steps: (a) contacting the sample with a fixative, thereby producing a fixed sample; (b) contacting the fixed sample with a hybridization solution, the hybridization solution comprising one or more a primary click-amplifying FISH (clampFISH) probes which hybridize to one or more regions of the target nucleic acid and adding a click chemistry agent that covalently locks the one or more primary clampFISH probes to the one or more regions of the target nucleic acid to form a primary sample; (c) contacting the primary sample with a set of secondary clampFISH probes that bind to the one or more primary clampFISH probes and adding a click chemistry agent that covalently locks the set of secondary clampFISH probes to the one or more primary clampFISH probes to form a secondary sample; (d) contacting the secondary sample with a set of tertiary clampFISH probes that bind to each secondary clampFISH probe and adding a click chemistry agent that covalently locks the set tertiary clampFISH probes to each secondary clampFISH probe to form a tertiary sample; (e) contacting the tertiary sample with a set of secondary clampFISH probes that bind to each tertiary clampFISH probes and adding a click chemistry agent that covalently locks the set secondary clampFISH probes to each tertiary clampFISH probe; and, (f) repeating steps (d) and (e) until a desired level of fluorescent signal of the labeled target nucleic acid is achieved; and detecting the fluorescent signal of the labeled target nucleic acid wherein the level of fluorescent signal is exponentially amplified.

In one embodiment, both steps of (1) contacting the secondary sample with a set of tertiary clampFISH probes that bind to each secondary clampFISH probe and adding a click chemistry agent that covalently locks the set tertiary clampFISH probes to each secondary clampFISH probe to form a tertiary sample (i.e. step (d)) ; and (2) contacting the tertiary sample with a set of secondary clampFISH probes that bind to each tertiary clampFISH probes and adding a click chemistry agent that covalently locks the set secondary clampFISH probes to each tertiary clampFISH probe (i.e. step (e)), are repeated 1, 2, 3, 4, 5, 6, 7, 8, 9 or 10 times. In some embodiments these steps are repeated for further rounds such as 11, 12, 13, 14, 15, 16, 17, 18, 19, 20 or more times.

In one embodiment, the methods of the invention comprise providing a sample. As discussed elsewhere herein, the sample may be derived or obtained from one or more eukaryotic cells, prokaryotic cells, bacteria, virus, exosome, liposome, and the like. In certain embodiments, the sample is obtained from a subject (e.g., a biological sample), including, for example, from a human, swine, or avian subject. In one embodiment, the sample is a cell. In another embodiment, the sample is a tissue sample. In one embodiment, the sample is a body sample, including, for example, blood, urine, skin, fat, saliva, and the like. In one embodiment, the sample is attached to a surface (e.g. a glass slide or a petri dish) or suspended in liquid solution.

In one embodiment, the methods comprise fixing the sample. The present invention includes the use of any compositions and methods for crosslinking or non-crosslinking fixation known in the art. In one embodiment, the sample is fixed using an aldehyde-based fixative. For example, in one embodiment, the method comprises fixing the sample using a fixative comprising formaldehyde or paraformaldehyde. In one embodiment, the sample is fixed using an alcohol-based fixative. For example, in one embodiment, the method comprises fixing the sample using a fixative comprising methanol or ethanol. Fixation of the sample may be done under any suitable conditions which results in the fixation of the sample. For example, in one embodiment, the sample is contacted with the fixative for about 1 second to about 5 hours. In one embodiment, the sample is contacted with the non-cross-linking fixative (e.g., methanol) for about 1 minute. In another embodiment, the sample is contacted with the non-crosslinking fixative for about 10 minutes. In one embodiment, the sample is contacted with the non-crosslinking fixative for about 1-10 minutes and any and all ranges therebetween. In one embodiment, the sample is contacted with the fixative at a temperature of about −80° C. to about 50° C. In some embodiments, the sample is contacted with the non-crosslinking fixative at a temperature of about −20° C. In other embodiments, the sample is contacted with the crosslinking fixative at room temperature (e.g., about 20-23° C.). In one embodiment, following incubation with the fixative, the sample is washed.

In one embodiment, the fixed sample is contacted with a hybridization solution comprising a probe concentration of about 1 uM to about 100 mM. In another embodiment, the fixed sample is contacted with a hybridization solution comprising a probe concentration of about 10 uM to about 10 mM. In yet another embodiment, the fixed sample is contacted with a hybridization solution comprising a probe concentration of about 100 uM to about 1 mM. In a further embodiment, the fixed sample is contacted with a hybridization solution comprising a probe concentration of about 200-500 uM. In another embodiment, the fixed sample is contacted with a hybridization solution comprising a probe concentration of about 400 uM. The hybridization solution may further comprise any additional suitable components known in the art. For example, in one embodiment, the hybridization solution comprises formamide, saline-sodium citrate, and dextran sulfate.

In one embodiment, the sample is contacted with the hybridization solution for at least about 24 hours. In one embodiment, the method comprises contacting the sample with the hybridization solution for at least about 12 hours. In one embodiment, the method comprises contacting the sample with the hybridization solution for at least about 6 hours. In one embodiment, the method comprises contacting the sample with the hybridization solution at least about 4 hours. In one embodiment, the method comprises contacting the sample with the hybridization solution at least about 2 hours. In one embodiment, the method comprises contacting the sample with the hybridization solution at least about 1 hour. In certain embodiments, the use of a sample contacted with a non-crosslinking fixative allows for the use of a higher concentration of probes thereby shortening the hybridization time. In one embodiment, the method comprises contacting the sample with the hybridization solution for less than about 10 minutes. In one embodiment, the method comprises contacting the sample with the hybridization solution for less than about 5 minutes. In one embodiment, the method comprises contacting the sample with the hybridization solution for less than about 1 minute. Thus, in the embodiments disclosed herein, the method comprises contacting the sample with the hybridization solution at least about 1 minute to at least about 24 hours and any and all ranges therebetween.

In one embodiment, the methods of the invention comprise contacting the sample with about 0.1-1000 μL of hybridization solution. In another embodiment, the methods of the invention comprise contacting the sample with about 1 μL of hybridization solution.

Hybridization of the probes to the sample may be performed in any suitable hybridization conditions known in the art. For example, in one embodiment, the sample is contacted with the hybridization solution at a temperature of about 0° C. to about 100° C. In one embodiment the sample is contacted with the hybridization solution at a temperature of about 37° C. In one embodiment, following incubation with the hybridization solution, the sample is washed. In certain embodiments, the wash times are about 1-60 min. In certain embodiments, the wash times are about 10-50 min. In certain embodiments, the wash times are about 20-40 min. In certain embodiments, the wash times are about 30 min. For example, in one embodiment, the washing of the sample comprises two or three separate 30 minute incubations with a wash solution. The wash solution may be any standard or suitable buffer or solution known in the art.

In certain embodiments, the sample is imaged and analyzed for the presence, location, or amount of one or more targets. Imaging of the sample may be done using any suitable imaging instrumentation and software systems known in art.

In certain embodiments, the clampFISH method of this invention is used to identify a splice junctions in the target nucleic acid where each arm of the primary clampFISH probe targets the nucleic acid region closest to the respective exons. In other embodiments, the clampFISH method is useful to identify alternatively spiced variants. In other embodiments, the clampFISH method is used to identify a mutation in the target nucleic acid.

In certain embodiments, the clampFISH method described herein allows for exponential amplification of the signal of the fluorescently target labeled nucleic acid. This exponential amplification allows therefore a high-throughput detection of the target(s) of interest using low magnification microscopy or flow cytometry. In some embodiments, low magnification microscopy can be performed at a magnification of about 60× or less, 40× or less, 20× or less, 10× or less, and 4× or less.

In certain embodiments, the clampFISH method described herein could be used in conjunction with expansion microscopy. Expansion microscopy is a method well known in the art where a sample is linked to a swellable polymer and physically expanded to enable a high-resolution microscopy using a low magnification microscope (U.S. patent application Ser. No. 15/098,799).

### Probes

In one embodiment, the methods of the invention comprise contacting the fixed sample with one or more primary clampFISH probe. In another embodiment, the one or more primary clampFISH probe is contacted with a set of secondary clampFISH probes, wherein the secondary clampFISH probes bind in 2:1 ratio to the one or more primary clampFISH probe. In another embodiment, the one or more primary clampFISH probe is contacted with a set of secondary clampFISH probes, wherein the secondary clampFISH probes bind in a n:1 ratio to the one or more primary clampFISH probe, wherein “n” corresponds to the number of binding sites on the primary probe. In another embodiment, the secondary clampFISH probes are in turn contacted with a set of tertiary clampFISH probes, wherein the tertiary clampFISH probes bind in 2:1 ratio to the secondary clampFISH probes. In yet another embodiment, the secondary clampFISH probes are contacted with a set of tertiary clampFISH probes, wherein the tertiary clampFISH probes bind in a n:1 ratio to the secondary clampFISH probes, wherein “n” corresponds to the number of binding sites on the secondary probe. In still another embodiment, the tertiary clampFISH probes are in turn contacted with a set of secondary clampFISH probes, wherein the secondary clampFISH probes bind in 2:1 ratio to the secondary clampFISH probes. In a further embodiment, the tertiary clampFISH probes are contacted with a set of secondary clampFISH probes, wherein the secondary clampFISH probes bind in a n:1 ratio to the tertiary clampFISH probes, wherein “n” corresponds to the number of binding sites on the tertiary clampFISH probes. In yet a further embodiment, the set of secondary and tertiary clampFISH probes comprises at least 2 probes. In some embodiments, the steps involving the contacting of the secondary or tertiary probes are repeated. As mentioned above herein, these steps are repeated for 1 to 20 or more rounds. The number of round is generally chosen by the user based upon the type of sample and the desired ultimate level of signal amplification.

In one embodiment, the fluorescent signal is amplified more than 10, more than 50, more than 100 and more than 500 folds as compared to a control labeled with a standard FISH or labeled with a primary, secondary and tertiary clampFISH probes but without repeating any of the above steps involving the contacting of the secondary or tertiary probes. In one embodiment, the signal is amplified about 4 folds per 2 rounds. In other embodiments, the fluorescent signal is amplified about 120 folds after 6 rounds, and about 500 folds after 10 rounds.

Probes useful in this invention may be DNA, RNA or mixtures of DNA and RNA. They may include non-natural nucleotides, and they may include non-natural internucleotide linkages. Non-natural nucleotides that increase the binding affinity of probes include 2′-O-methyl ribonucleotides, for example.

The lengths of the clampFISH probes useful in this invention can be about 40-300 nucleotides. Preferred lengths of the probes are in the range of about 100-200 nucleotides, more preferably 125-175 nucleotides. In certain embodiments, the probes are about 150 nucleotides long. In one embodiment, the clampFISH probes comprise a left binding arm, a left adapter, a backbone, a right adapter and a right binding arm. In some embodiments, the binding arms are each about 15 nucleotides long and the adapter are each about 10 nucleotides long. In some embodiments, the backbone is about 100 nucleotides long.

In one embodiment, the nucleic acid of interest (RNA or DNA) are targeted by clampFISH probe sets containing one or more ClampFISH probe, each targeting a region of the target nucleic acid. In some instances the targeted region is 30 nucleotides long and is bound by 2 adjacent 15 nucleotides long ClampFISH binding arms (the left and right one). In other instances the target region is larger or smaller than 30 nucleotides long (e.g. 14 nucleotides long) and is bound by 2 adjacent ClampFISH binding arms having a total length equal or less to the target region (e.g. 7 nucleotides long left and right ClampFISH binding arms targeting a 14 nucleotides long region). In one embodiment, each probe of the primary, secondary and tertiary clampFISH probes binds to a different region of their respective nucleic acid target. In one embodiment, the primary, secondary and tertiary clampFISH probes are complementary through their 3′ and 5′ ends (as refer to as binding arms) to two regions of their respective nucleic acid target.

In one embodiment, the 3′ and 5′ ends of the clampFISH probes comprise an azide and an alkyne group respectively. Inversely, in another embodiment, the 3′ and 5′ ends of the clampFISH probes comprise an alkyne and an azide group respectively. In another embodiment, the internal region of the clampFISH probes comprises at two separate locations an azide and an alkyne group. In one embodiment, a click chemistry agent catalyzes an azide-alkyne cycloaddition thereby covalently locking the primary, secondary and tertiary clampFISH probes around their respective nucleic acid target. In one embodiment, a click chemistry agent connects the azide and alkyne groups, located at the 3′ and 5′ ends of the primary, secondary and tertiary clampFISH probes around their respective nucleic acid target. In one embodiment, a click chemistry agent connects the azide and alkyne groups, located internally within the primary, secondary and tertiary clampFISH probes, around their respective nucleic acid target. Click chemistry, as described in further details below herein, is a cycloaddition well known in the art where pairs of functional groups (e.g. alkyne and an azide) rapidly and selectively react and couple with each other. In one embodiment, the click chemistry reaction of this invention is catalyzed by a copper(I), a copper(II) or a ruthenium. In another embodiment, the click chemistry agent used herein comprises a copper(II), BTTAA ligand, and sodium ascorbate. In a further embodiment, the click chemistry reaction is a copper-free strain promoted azide alkyne cycloaddition (SpAAC) comprising a cyclooctyne that undergoes azide-alkyne Huisgen cycloaddition without the need of a copper catalyzer.

In a further embodiment, a fluorophore can be attached to the clampFISH probes of this invention. The fluorophore can be attached at any position, including, without limitation, attaching a fluorophore to one end of a probe, preferably to the 3′ end. The clampFISH probes may be included in a hybridization solution that contains the probes in excess.

The clampFISH probes of this invention may be designed to specifically bind to any target nucleic acid, including DNA, RNA, mRNA, non-coding RNA, microRNA, siRNA, and the like. In some embodiments, the target nucleic acid can comprise an alternatively spliced or a mutational variant.

In one embodiment, the clampFISH probes are useful for a multiplex assay. In certain embodiments, more than one type of probe is used. For example, in certain embodiments, about 1-1000 different probes are used. In one embodiment, each of the different probes are labeled with a similar of different fluorophore and are hybridized simultaneously to a target sequence of a nucleotide molecule, such as an RNA molecule. In some embodiments, the probes are not labeled and comprise unique backbone regions that bind secondary and tertiary probes. Ultimately the terminating probe (e.g. a tertiary probe) is labeled with a fluorophore using for instance a single molecule fluorescent in situ hybridization (smFISH) probe. In certain embodiments, the number of probes can range from 4-100, from 10-80, from 15-70, or from 20-60. A fluorescent spot is created that can be detected from the combined fluorescence of the multiple probes. The probes can be non-overlapping, meaning that the region of the target sequence to which each probe hybridizes is unique (or at least non-overlapping). Probes in a set of 2 or more for a selected target sequence can be designed to hybridize adjacently to one another or to hybridize non-adjacently, with stretches of the target sequence, from one nucleotide to a hundred nucleotides or more, not complementary to any of the probes.

In some embodiments, a single cell can be probed simultaneously for multiple nucleic acid target sequences, either more than one target sequence of one nucleic acid molecule, or one or more sequences of different nucleic acid molecules. Additionally, one target sequence of an nucleic acid molecule can be probed with more than one set of probes, wherein each set is labeled with a distinguishable fluorophore, and the fluorophores are distinguishable.

Methods of the present invention may also include determining if one or more fluorescent signal spots representing a target sequence is present. Methods according to the present invention also allow counting fluorescent signal spots of a given color corresponding to a given nucleic acid species. When it is desired to detect more than one nucleic acid species, different sets of probes labeled with distinct fluorophores can be used in the same hybridization mixture. The exponentially amplified fluorescent signal spots of this invention can be detected by using any microscopic or flow cytometry methods known in the art.

### Kits

In one embodiment, the present invention provides a kit, generally comprising primary, secondary and tertiary click-amplifying FISH (clampFISH) probes and a click chemistry agent for signal amplification and detection of nucleic acids in a sample and instructions for use thereof. In one embodiment, the click chemistry agent comprises copper (II), BTTAA ligand, and sodium ascorbate.

In some embodiments, the kit further comprises solutions, fixatives, and an instruction manual for performing any of the methods contemplated herein.

In some embodiments, the methods and kits of this invention may be utilized in diagnostic, prognostic, and screening methods. For example, in certain embodiments, the present methods are used to detect the presence, location, or amount one or more biomarker associated with a disease or disorder.

### Target Nucleic Acid Sample

As contemplated herein, the present invention may be used in the analysis of sample for which nucleic acid analysis may be applied, as would be understood by those having ordinary skill in the art. For example, in one embodiment, the sample comprises at least one target nucleic acid, whose presence, location, or amount is desired to be investigated. For example, in certain embodiments, the nucleic acid can be mRNA. However, it should be appreciated that there is no limitation to the type of nucleic acid sample, which may include without limitation, any type of RNA, cDNA, genomic DNA, fragmented RNA or DNA and the like. In certain embodiments, the nucleic acid sample comprises at least one of messenger RNA, intronic RNA, exonic DNA, and non-coding RNA. The nucleic acid may be prepared for hybridization according to any manner as would be understood by those having ordinary skill in the art. It should also be appreciated that the sample may be an isolated nucleic acid sample, or it may form part of a lysed cell, or it may be an intact living cell. Samples may further be individual cells, or a population of cells, such as a population of cells corresponding to a particular tissue. Samples may also be a tissue section. It should be appreciated that there is no limitation to the size or type of sample, provided the sample includes at least one nucleic acid therein. For example, the sample may be derived or obtained from one or more eukaryotic cells, prokaryotic cells, bacteria, virus, exosome, liposome, and the like. In certain embodiments, a sample is fixed. For example, in one embodiment, a living cell or tissue is provided and fixed prior to application of one or more probes. In one embodiment, the sample is fixed using a crosslinking fixative (such as an aldehyde-based fixative). In other embodiments, the sample is fixed using a non-crosslinking fixative (such as an alcohol-based fixative).

### Click Chemistry

The present exponential fluorescent amplification of nucleic acids, via the clampFISH probes, circumvent enzyme-based amplification schemes by relying on a series of click chemistry reactions which are key for this invention.

In one embodiment, a click chemistry agent connects the 3′ and 5′ azide/alkyne ends of the primary, secondary and tertiary clampFISH probes around the their respective nucleic acid target. In one embodiment, the click chemistry is catalyzed by a copper(I), a copper(II) or a ruthenium.

Azides and terminal alkynes can undergo Copper(I)-catalyzed Azide-Alkyne Cycloaddition (CuAAC) at room temperature. In this type of cycloaddition, also known as click chemistry, organic azides and terminal alkynes react to give 1,4-regioisomers of 1,2,3-triazoles. Examples of “click” chemistry reactions are described by Sharpless et al. (U.S. patent application Ser. No. 10/516,671), which developed reagents that react with each other in high yield and with few side reactions in a heteroatom linkage (as opposed to carbon-carbon bonds) in order to create libraries of chemical compounds. As described herein, click chemistry is used in the methods for labeling nucleic acids.

In some embodiments, the copper used as a catalyst for the click chemistry reaction is in the Cu (I) reduction state. This cycloaddition can also be conducted in the presence of a metal catalyst and a reducing agent. In certain embodiments, copper can be provided in the Cu (II) reduction state (for example, as a salt, such as but not limited to Cu(NO₃)₂ Cu(OAc)₂ or CuSO₄), in the presence of a reducing agent wherein Cu(I) is formed in situ by the reduction of Cu(II). Such reducing agents include, but are not limited to, ascorbate, Tris(2-Carboxyethyl) Phosphine (TCEP), 2,4,6-trichlorophenol (TCP), NADH, NADPH, thiosulfate, metallic copper, quinone, hydroquinone, vitamin K₁, glutathione, cysteine, 2-mercaptoethanol, dithiothreitol, Fe²⁺, Co²⁺, or an applied electric potential. In other embodiments, the reducing agents include metals selected from Al, Be, Co, Cr, Fe, Mg, Mn, Ni, Zn, Au, Ag, Hg, Cd, Zr, Ru, Fe, Co, Pt, Pd, Ni, Rh, and W. In other embodiments, the copper used as a catalyst for the click chemistry reaction is in the Cu (II) state and is reduced to Cu(I) with sodium ascorbate.

The present copper-catalyzed azide-alkyne cycloadditions for labeling nucleic acids can be performed in water and a variety of solvents, including mixtures of water and a variety of (partially) miscible organic solvents including alcohols, dimethyl sulfoxide (DMSO), dimethyl formamide (DMF), tert-butanol (tBuOH) and acetone.

Certain metal ions are unstable in aqueous solvents, by way of example Cu(I), therefore stabilizing ligands/chelators can be used to improve the reaction. In certain embodiments at least one copper chelator is used in the methods described herein, wherein such chelators bind copper in the Cu (I) state. In certain embodiments at least one copper chelator is used in the methods described herein. In certain embodiments, the copper (I) chelator is a 1,10 phenanthroline-containing copper (I) chelator. Non-limiting examples of such phenanthroline-containing copper (I) chelators include, but are not limited to, bathophenanthroline disulfonic acid (4,7-diphenyl-1,10-phenanthroline disulfonic acid) and bathocuproine disulfonic acid (BCS; 2,9-dimethyl-4,7-diphenyl-1,10-phenanthroline disulfonate). Other chelators used in such methods include, but are not limited to, N-(2-acetamido)iminodiacetic acid (ADA), pyridine-2,6-dicarboxylic acid (PDA), S-carboxymethyl-L-cysteine (SCMC), trientine, tetra-ethylenepolyamine (TEPA), NNNN-tetrakis(2-pyridylmethyl)ethylenediamine (TPEN), EDTA, neocuproine, N-(2-acetamido)iminodiacetic acid (ADA), pyridine-2,6-dicarboxylic acid (PDA), S-carboxymethyl-L-cysteine (SCMC), tris-(benzyl-triazolylmethyl)amine (TBTA), or a derivative thereof. Most metal chelators, a wide variety of which are known in the art, are known to chelate several metals, and thus metal chelators in general can be tested for their function in 1,3 cycloaddition reactions catalyzed by copper. In certain embodiments, histidine is used as a chelator, while in other embodiments glutathione is used as a chelator and a reducing agent.

The concentration of the reducing agents used in the “click” chemistry reaction described herein can be in the micromolar to millimolar range. In certain embodiments the concentration of the reducing agent is from about 100 micromolar to about 100 millimolar. In other embodiments the concentration of the reducing agent is from about 10 micromolar to about 10 millimolar. In other embodiments the concentration of the reducing agent is from about 1 micromolar to about 1 millimolar. In yet other embodiments, the concentration of the reducing agent is 2.5 millimolar.

The concentration of a copper chelator used in the “click” chemistry reaction described herein can be determined and optimized using methods well known in the art. In certain embodiments, the chelator concentrations used in the methods described herein is in the micromolar to millimolar range, by way of example only, from 1 micromolar to 100 millimolar. In certain embodiments the chelator concentration is from about 10 micromolar to about 10 millimolar. In other embodiments the chelator concentration is from about 50 micromolar to about 10 millimolar. In other embodiments the chelator, can be provided in a solution that includes a water miscible solvent such as, alcohols, dimethyl sulfoxide (DMSO), dimethyl formamide (DMF), tert-butanol (tBuOH) and acetone. In other embodiments the chelator, can be provided in a solution that includes a solvent such as, for example, dimethyl sulfoxide (DMSO) or dimethylformamide (DMF).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Materials and Methods

### Cell Culture

WM983b cells and WM983b-GFP-NLS cells (a human metastatic melanoma cell line) were cultured in tumor specialized media containing 2% FBS. The WM983b-GFP-NLS contains EGFP fused to a nuclear localization signal driven by a cytomegalovirus promoter that was stably transfected into the parental cell line.

### Clamp Probe Design and Synthesis

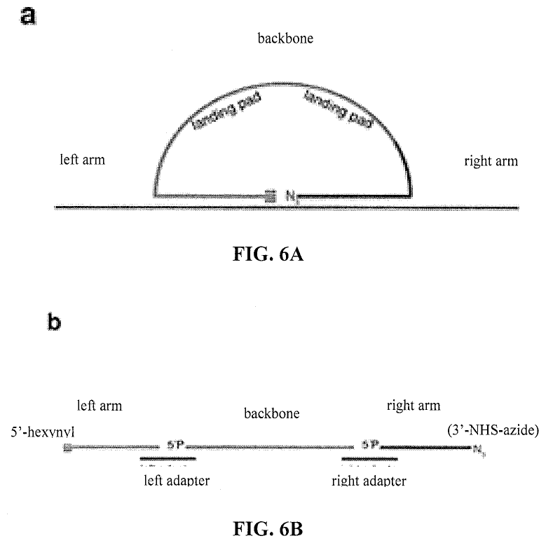

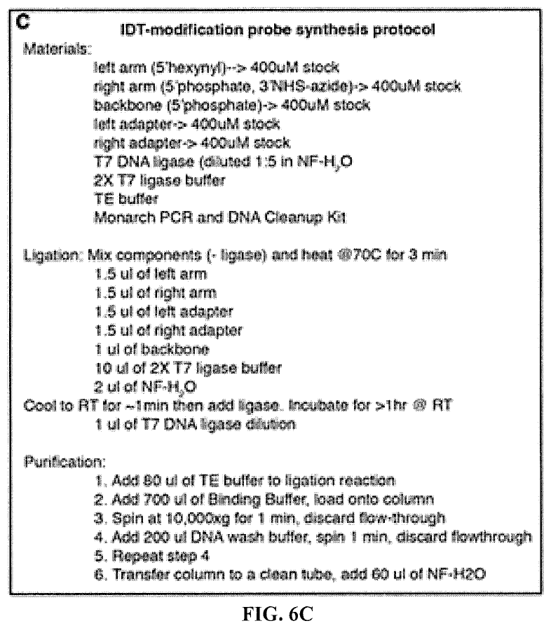

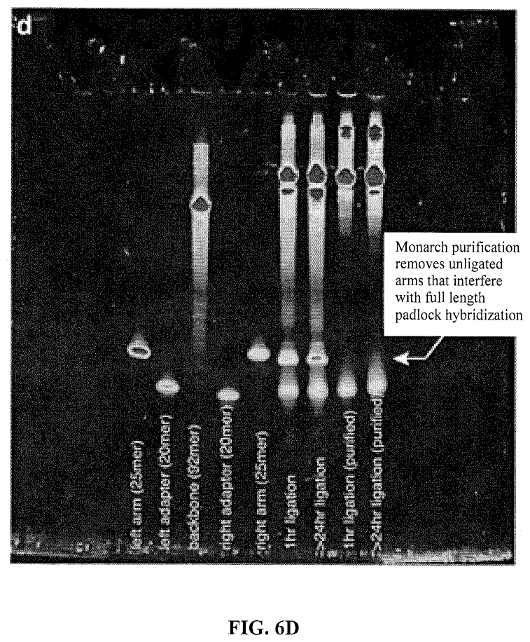

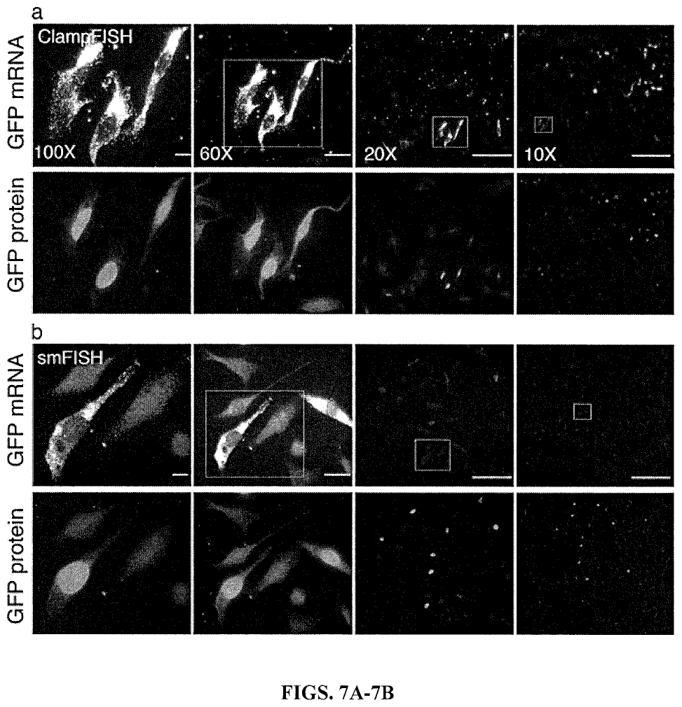

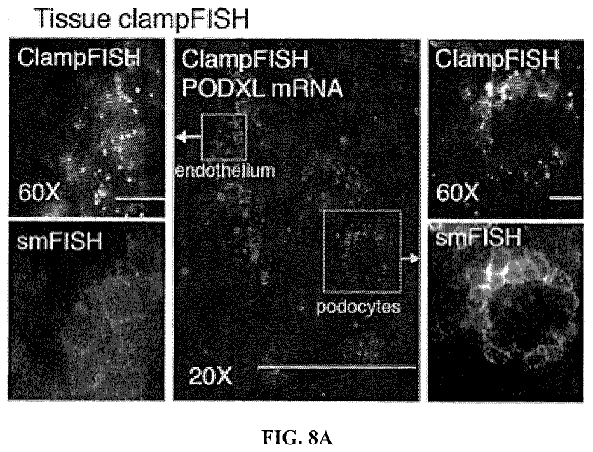

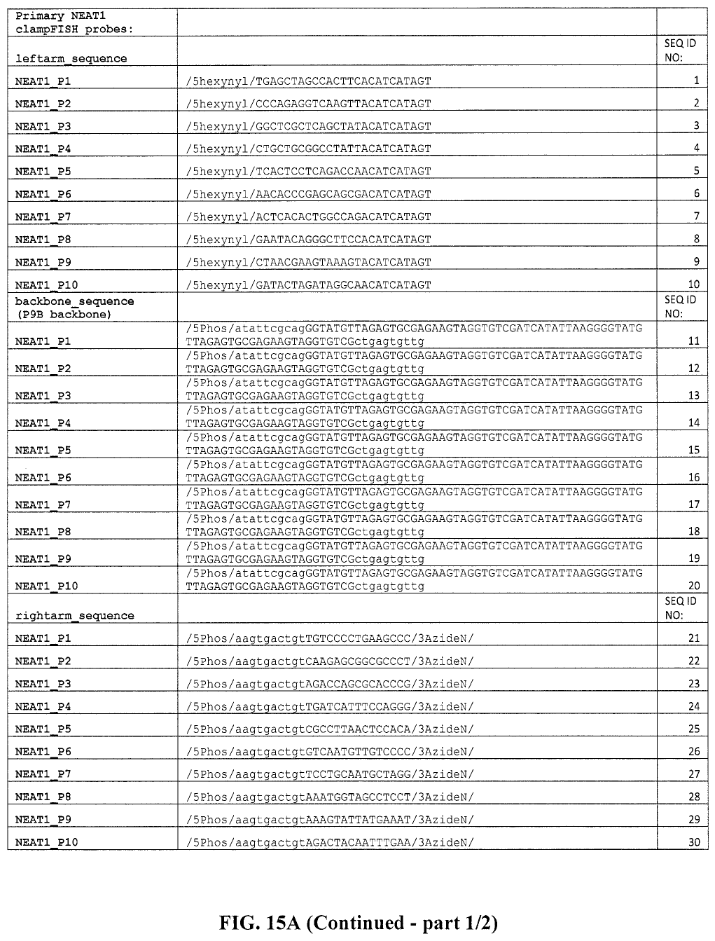

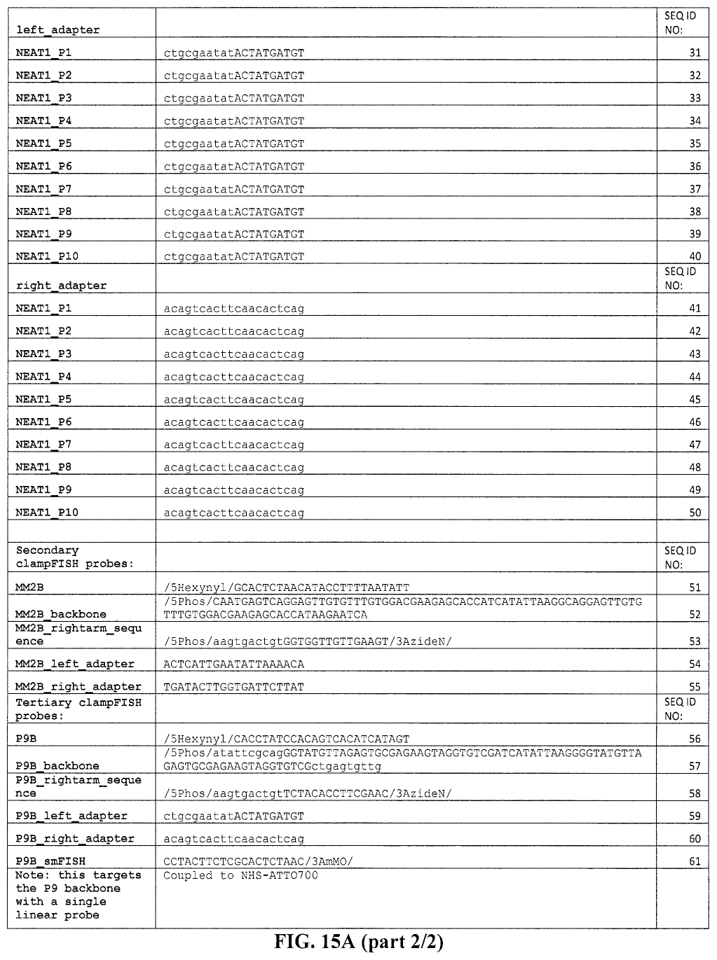

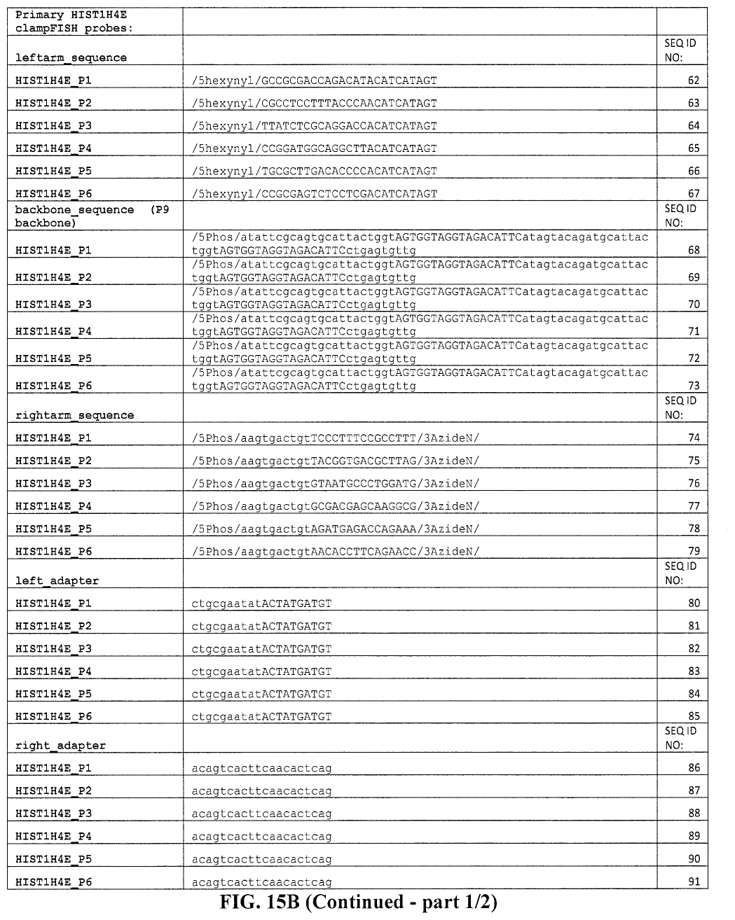

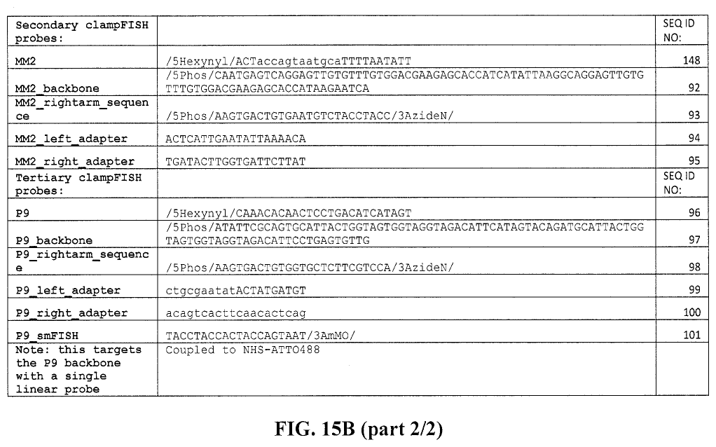

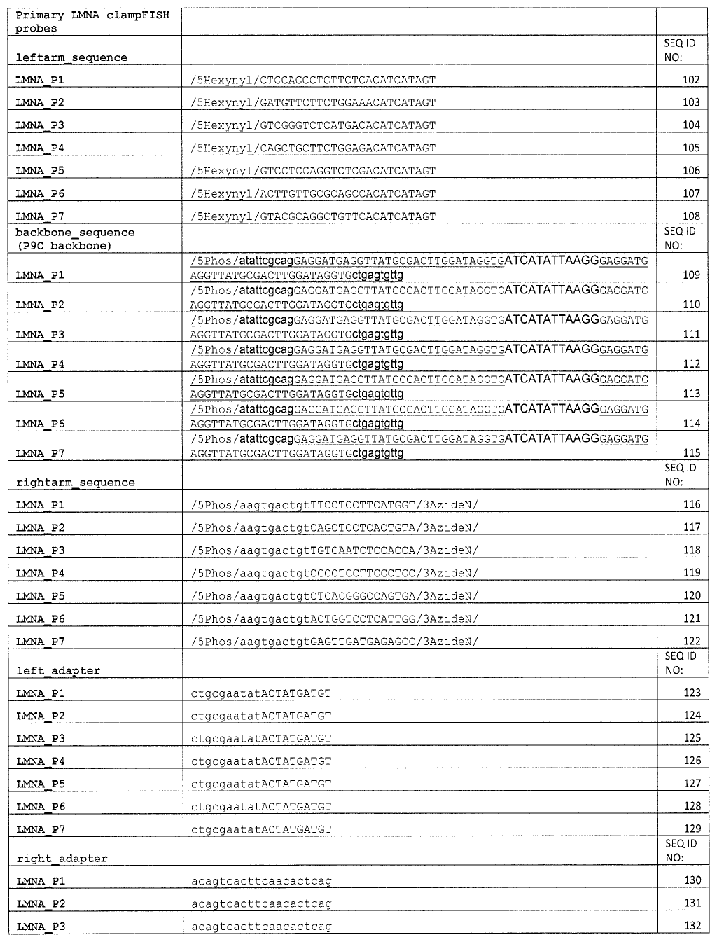

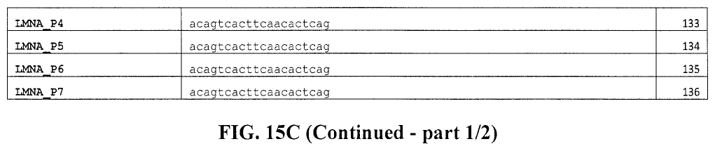

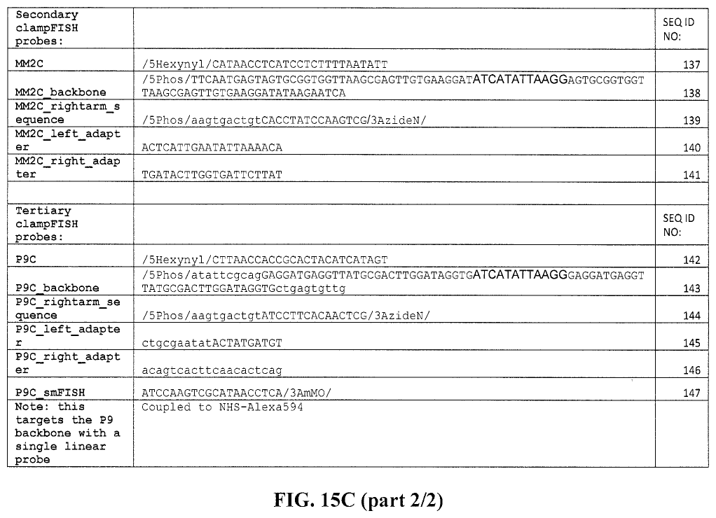

Clamp probes are 150 nt long (15 mer left RNA binding arm, 10 nt left adapter, 100 mer backbone, 10 nt right adapter, 15 mer right RNA binding arm). RNAs are targeted by probe sets containing one or more Clamp probes, each targeting a 30 nt region of RNA (2 adjacent 15 mer binding arms). The binding regions were chosen with approximately 40% GC content as well as minimal repetitive regions. The backbones were designed so as to minimize secondary structure (using the mFold web server at unafold.rna.albany.edu/?q=mfold). Modified DNA oligonucleotides were ordered from Integrated DNA Technologies (IDT) as standard DNA oligonucleotides with modifications (5′-phosphate on the backbone, 3′-azide and 5′-phosphate for the right arm and 5′-hexynyl for the left arm). Strands were resuspended in nuclease free water, at a working stock concentration of 400 uM. The left arm (30 uM), backbone (20 uM) and right arm (30 uM) are brought together using adapter probes (30 uM each) and enzymatically ligated using 600 U of T7 DNA ligase in a 10 ul reaction for a minimum of 1 hour at room temperature. Following ligation, the probes were purified using Monarch purification columns (New England Biolabs) and eluted in 4× the starting volume to make the working dilution. For a schematic protocol and probe sequences, see FIGS. 6A-6D and FIGS. 15A-15C (SEQ ID Nos: 1-147).

### ClampFISH Procedure for Cultured Cells

Cells were grown on glass coverslides until ˜70% confluent. They were washed twice with 1× PBS, then fixed for 10 minutes with 4% formaldehyde/1× PBS at room temperature. The formaldehyde was aspirated, and cells were rinsed twice with 1× PBS prior to adding 70% ethanol for storage at 4° C. Then cells were incubated for at least 4 hours at 37° C. in hybridization buffer (10% dextran sulfate, 2×SSC, 20% formamide) and 1 ul of the working dilution of the primary ClampFISH probe. Two washes were performed in wash buffer (2×SSC, 10% formamide), each consisting of a 30-min incubation at 37° C. The cells were then incubated for at least 2 hours at 37° C. in hybridization buffer (10% dextran sulfate, 2×SSC, 20% formamide) and 1 ul of the working dilution of the secondary ClampFISH probe and repeated the washes. After the second wash, the ‘click’ reaction was performed. A solution containing 75 μM CuSO₄.5H2O premixed with 150 μM BTTAA ligand and 2.5 mM sodium ascorbate (made fresh and added to solution immediately before use) in 2×SSC was added to the samples, and these were incubated for 30 min at 37° C. The samples were rinsed briefly with wash buffer, then this protocol was repeated by alternating between secondary and tertiary ClampFISH probes until reaching the desired level of amplification. After the final wash, the samples were rinsed once with 2× SCC/DAPI and once with anti-fade buffer (10 mM Tris (pH 8.0), 2×SSC, 1% w/v glucose). Finally, the samples were mounted for imaging in an anti-fade buffer with catalase and glucose oxidase (Raj et al., 2008) to prevent photobleaching.

### ClampFISH for Flow Cytometry

ClampFISH for flow cytometry was performed as described above however the cells were kept in suspension. Wash buffer and 2×SSC were supplemented with 0.25% triton-X, and the ClampFISH hybridization buffer was supplemented with the following blocking reagents: 1 μg/μl yeast tRNA (Invitrogen P/N 54016), 0.02% w/v Bovine Serum Albumin, 100 ng/μl sonicated salmon sperm DNA (Agilent, 201190-81).

### ClampFISH for Expansion Microscopy

Acryloyl-X, SE (6-((acryloyl)amino)hexanoic acid, succinimidyl ester, here abbreviated AcX; Thermo-Fisher) was resuspended in anhydrous DMSO at a concentration of 10 mg/mL, aliquoted and stored frozen in a desiccated environment. Label-IT® Amine Modifying Reagent (Mirus Bio, LLC) was resuspended in the provided Mirus Reconstitution Solution at 1 mg/ml and stored frozen in a desiccated environment. To prepare LabelX, 10 μL of AcX (10 mg/mL) was reacted with 100 μL of Label-IT® Amine Modifying Reagent (1 mg/mL) overnight at room temperature with shaking. LabelX was subsequently stored frozen (−20° C.) in a desiccated environment until use.

Fixed cells were washed twice with 1× PBS and incubated with LabelX diluted to 0.002 mg/mL in MOPS buffer (20 mM MOPS pH 7.7) at 37° C. for 6 hours followed by two washes with 1× PBS. Monomer solution (1× PBS, 2 M NaCl, 8.625% (w/w) sodium acrylate, 2.5% (w/w) acrylamide, 0.15% (w/w) N,N′-methylenebisacrylamide) was mixed, frozen in aliquots, and thawed before use. Prior to embedding, monomer solution was cooled to 4° C. to prevent premature gelation. Concentrated stocks (10% w/w) of ammonium persulfate (APS) initiator and tetramethylethylenediamine (TEMED) accelerator were added to the monomer solution up to 0.2% (w/w) each. 100 uL of gel solution specimens were added to each well of a Lab Tek 8 chambered coverslip and transferred to a humidified 37° C. incubator for two hours.

Proteinase K (New England Biolabs) was diluted 1:100 to 8 units/mL in digestion buffer (50 mM Tris (pH:sunglasses:, 1 mM EDTA, 0.5% Triton X-100, 0.8 M guanidine HCl) and applied directly to gels in at least ten times volume excess. The gels were then incubated in digestion buffer for at least 12 hours. Gels were then incubated with wash buffer (10% formamide, 2×SSC) for 2 hours at room temperature and hybridized with RNA FISH probes in hybridization buffer (10% formamide, 10% dextran sulfate, 2×SSC) overnight at 37° C. Following hybridization, samples were washed twice with wash buffer, 30 minutes per wash, and washed 4 times with water, 1 hr per wash, for expansion. Samples were imaged in water with 0.1 ug/mL DAPI.

### ClampFISH for Mouse Tissues

Tissues were dissected and immediately embed in OCT, then were frozen using liquid nitrogen. Samples were stored at −80° C. or directly processed. Using a cryostat, 5 μm tissue sections were cut at −20° C. and the slices were transferred to charged slides. The slides were washed briefly in PBS to remove excess OCT, then immersed in 4% paraformaldehyde for 10 min at room temperature. Following fixation, the slides were transferred to 70% ethanol for permeabilization for at least 12 hours, or for long-term storage. To begin the clampFISH procedure, the slides were transferred to wash buffer for 3 minutes to equilibrate, then 500 ul of 8% SDS were added to the top of the flat slide for 1 minutes for tissue clearing. Then the samples were transferred to wash buffer, and proceeded with the regular clampFISH procedure.

### Imaging

Each samples was imaged on a Nikon Ti-E inverted fluorescence microscope a cooled CCD camera (Andor iKon 934). For 100× imaging, z-stacks (0.3 μm spacing between stacks) of stained cells were acquired. The filter sets used herein were 31000v2 (Chroma), 41028 (Chroma), SP102v1 (Chroma),17 SP104v2 (Chroma) and SP105 (Chroma) for DAPI, Atto 488, Cy3, Atto 647N/Cy5 and Atto 700, respectively. A custom filter set was used for Alexa 594 (Omega). Exposure times varied depending on the dyes and degree of amplification used. Typically, ClampFISH imaging was done at a 300 ms exposure and smFISH was done at 2-3 s exposure.

Image analysis

First images were segmented and thresholded using a custom Matlab software suite (downloadable at bitbucket.org/arjunrajlaboratory/rajlabimagetools/wiki/Home). Segmentation of cells was done manually by drawing a boundary around non-overlapping cells. The software then fitted each spot to a two-dimensional Gaussian profile specifically on the Z-plane on which it arose in order to ascertain subpixel-resolution spot locations. Colocalization took place in two stages: In the first stage, guide spots searched for the nearest-neighbor SNP probes within a 2.5-pixel (360-nm) window. The median displacement vector field was determined for each match and subsequently was used to correct for chromatic aberrations. After this correction, a more stringent 1.5-pixel (195-nm) radius was used to make the final determination of colocalization. In order to test random colocalization due to spots occurring by chance, the present images were taken and the guide channel was shifted by adding 5 pixels (1.3 μm) to the X and Y coordinates and then colocalization analysis was performed.

### Reproducible Analyses

Scripts for all analyses presented herein, including all data extraction, processing, and graphing steps are freely accessible online at www.dropbox.com/sh/qj2hanxe3hs14ry/AACx_3IO9LVVYX4DhKWxqoKla?dl=0

The results of the experiments are now described.

### Example 1: Design and Validation of clampFISH Technology

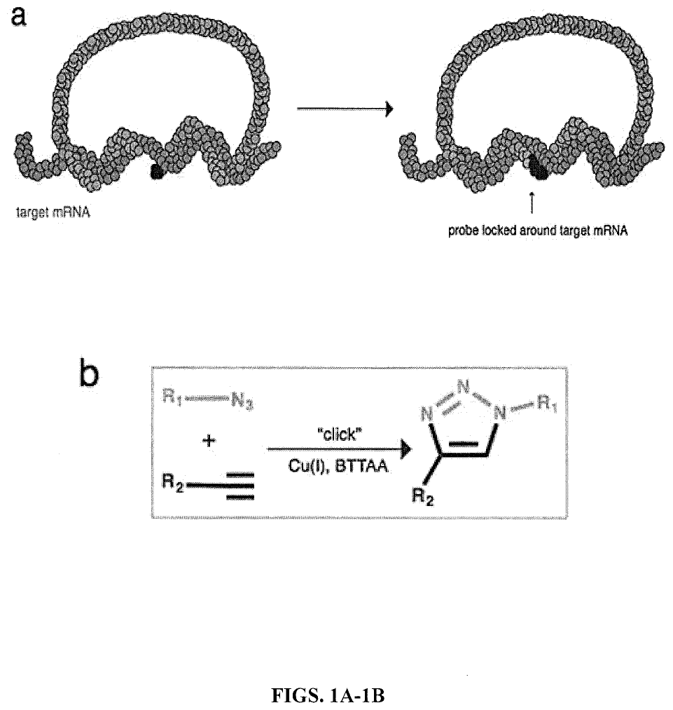

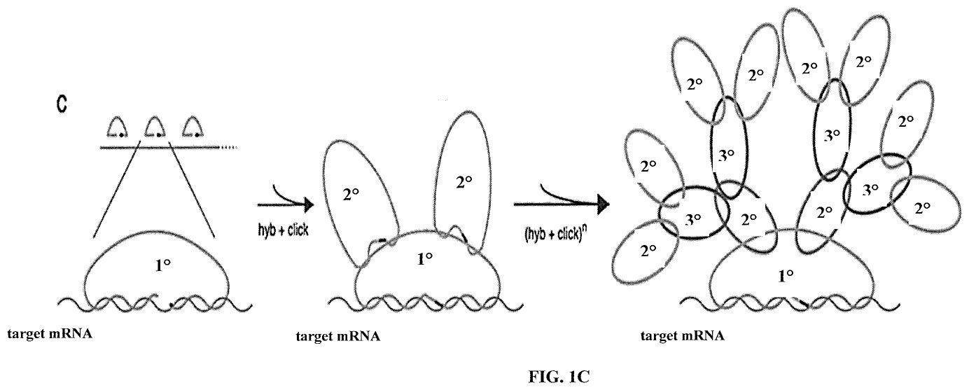

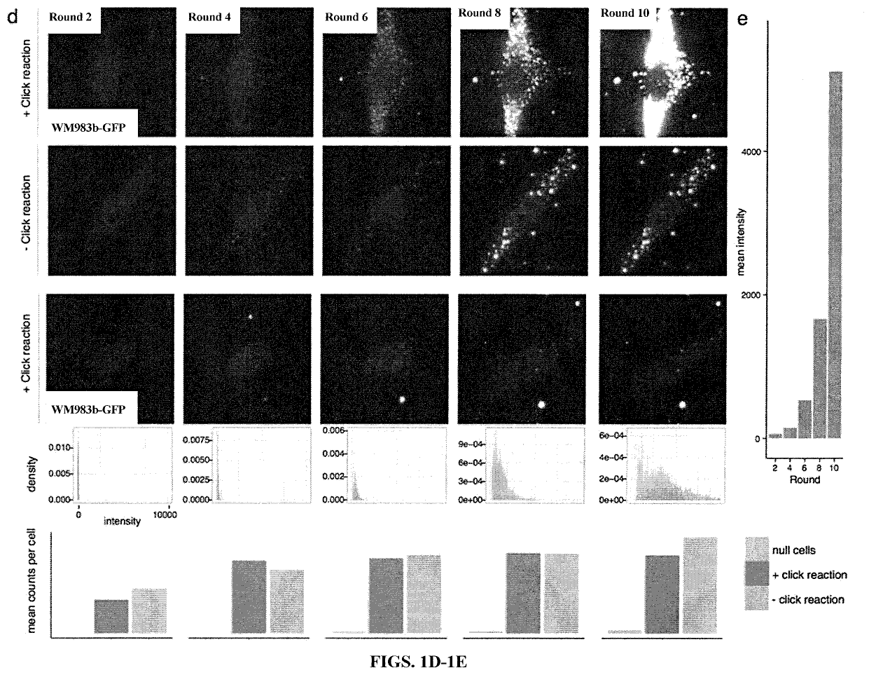

The main goal of the present invention is to amplify the fluorescent signal of individual RNAs in a manner that was both highly sensitive (high gain amplification) and highly specific (low background). To satisfy these conditions, molecular inversion (“padlock”) probes (MIP) that are complementary to their target nucleic acids at the 5′ and 3′ ends were used (Nilsson et al., Science 265, 2085-2088 (1994)). This class of oligonucleotide probe topologically wraps around the target sequence in a helical manner, and so can be covalently “locked” in place (FIG. 1A and FIGS. 6A-6D). This locking property was thought to allow the probes to survive repeated liquid handling in conditions stringent enough to limit the background. Traditionally, these probes have been locked around their target sequence using enzymes such as DNA ligase, but as mentioned previously herein, they often suffer in sensitivity because enzymes are bulky and often have a hard time finding their target in the cross-linked milieu of a fixed cell. A click chemistry strategy (copper(I)-catalyzed azide-alkyne cycloaddition, CuAAC (Besanceney-Webler et al., Angew. Chem. Int. Ed Engl. 50, 8051-8056 (2011)); FIG. 1B) was conceived herein to connect the 5′ and 3′ ends of the probe around the target RNA sequence to lock the probes in place without the need for any enzymes. Next the goal was to amplify signal from 10 primary click-amplifying FISH (clampFISH) probes. To achieve amplification from these primary clampFISH probes, a set of secondary, fluorescent, clampFISH probes that bound twice to each primary probe was designed. To these secondary probes, a set of tertiary probes was bound again in a 2:1 ratio. In a subsequent round, the secondary probes were bound again in a 2:1 ratio to the tertiary probes and so on, thus providing exponential amplification (FIG. 1C).

To demonstrate that clampFISH amplification was possible, a GFP mRNA in a human melanoma cell line (WM983b) stably expressing GFP was first targeted and amplified using 10 primary clampFISH probes. Stringent hybridization conditions were used, specifically, a higher concentration of formamide than is traditionally used for smFISH, to limit nonspecific probe binding. As the number of rounds progressed, the intensity of the signal as measured by fluorescence microscopy increased, roughly 4 fold per 2 rounds as expected (FIG. 1D), while the average number of spots per cell remained roughly constant. At round 10, the mean signal was amplified approximately 500 fold (FIG. 1E). To demonstrate signal specificity, the same clampFISH detection and amplification were performed on the same cell line but without GFP. Very few spots were detected in these cells, showing that the signals were specific to the target (FIG. 1D). To assess whether the click reaction aided in the amplification process, the same experiment was performed in the absence of the click-ligation of the clampFlSH probes. Although the number of spots detected per cell were similar, a lower mean signal intensity as well as a lack of uniformity in spot intensity were observed, demonstrating that the click reaction facilitated uniform amplification of primary clampFISH signal.

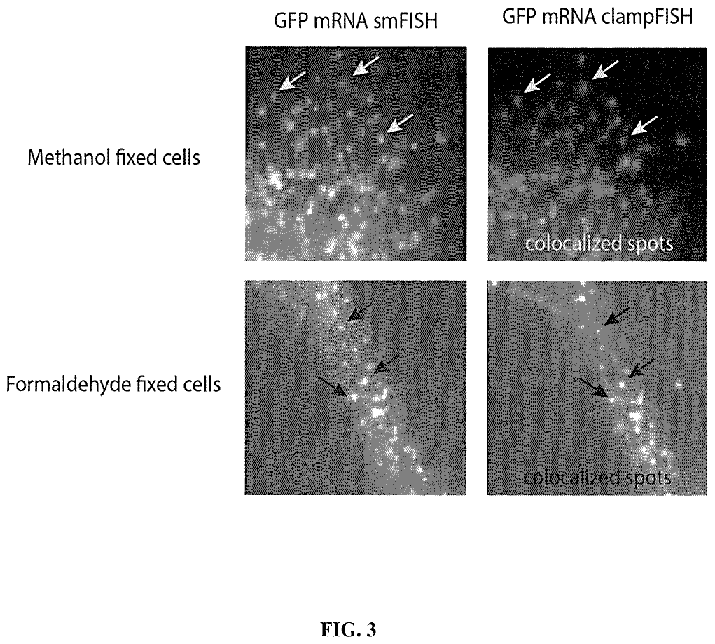

Additionally, the feasibility of clampFlSH technology was assessed in a sample (e.g. live cells) fixed using an alcohol-based fixative (i.e. methanol). Live cells are washed with PBS, then incubated with cold methanol for 10 minutes, then stored in 70% ethanol. These cells are then hybridized with clampFlSH probes as described elsewhere herein. As shown in FIG. 3, clampFlSH technology can be used on methanol fixed cells as well as formaldehyde fixed cells. GFP clampFlSH probes were used and successfully bind to GFP mRNA target in both methanol fixed cells and formaldehyde fixed cells. The arrows indicate clampFlSH signal colocalized with GFP smFISH using both fixation methods.

### Example 2: Applications of clampFlSH Amplification of RNA

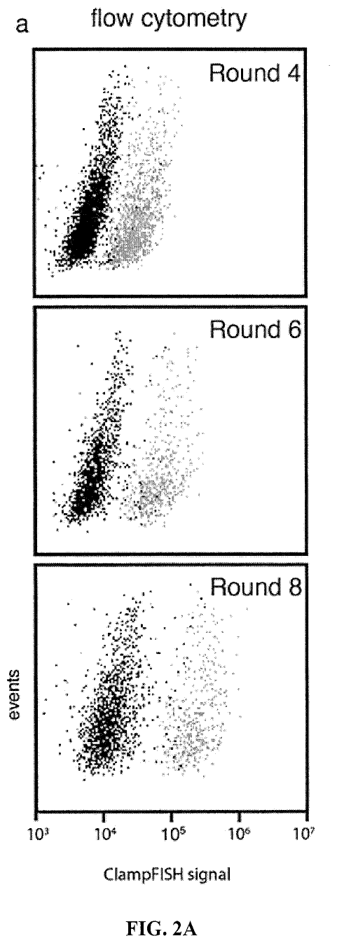

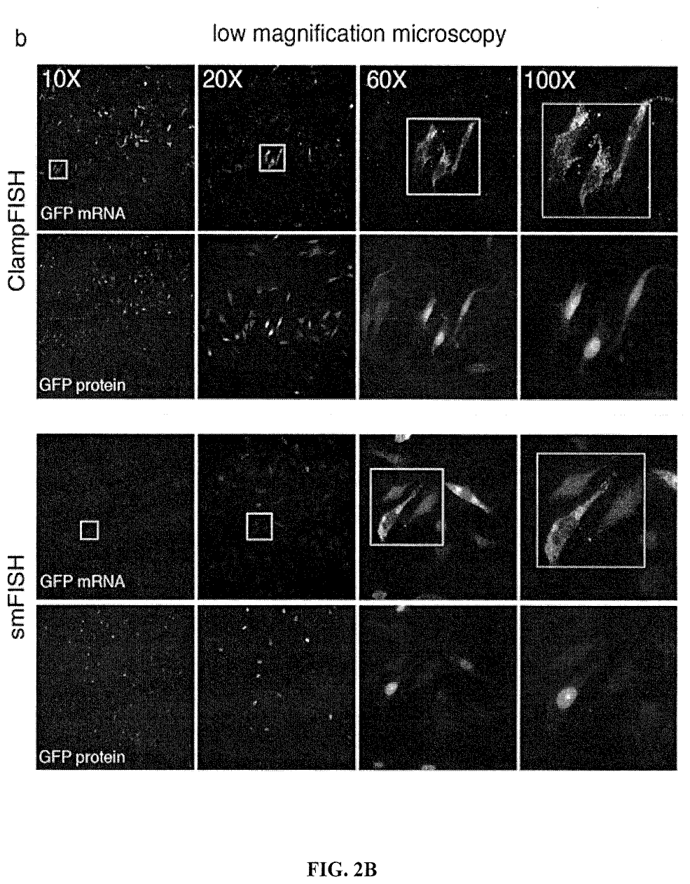

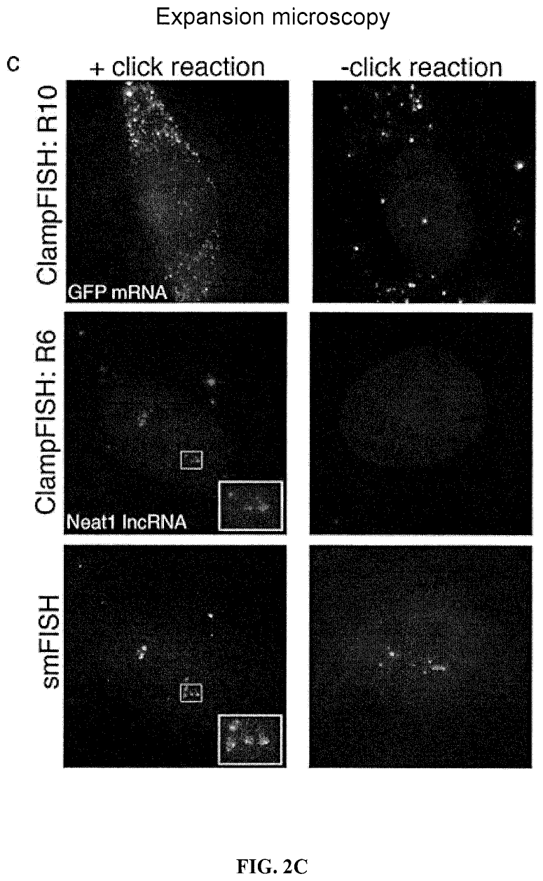

Next, the application of the present clampFISH invention revealed the ability of separating cells by flow cytometry based on their RNA expression, an application for which single molecule RNA FISH typically does not produce enough signal (Klemm et al., Nat. Methods 11, 549-551 (2014)). ClampFISH was applied to a mixed population of WM983b cells with and without GFP expression and analyzed the separation by flow cytometry (FIG. 2A). A separation of positive cells was detected as early as 4 rounds of amplification, and approximately a 4-fold increase in fluorescence intensity (and corresponding shift) in the GFP positive population was observed with every 2 rounds of amplification thereafter. The negative cells did not shift with the increasing rounds of amplification (FIG. 2A).

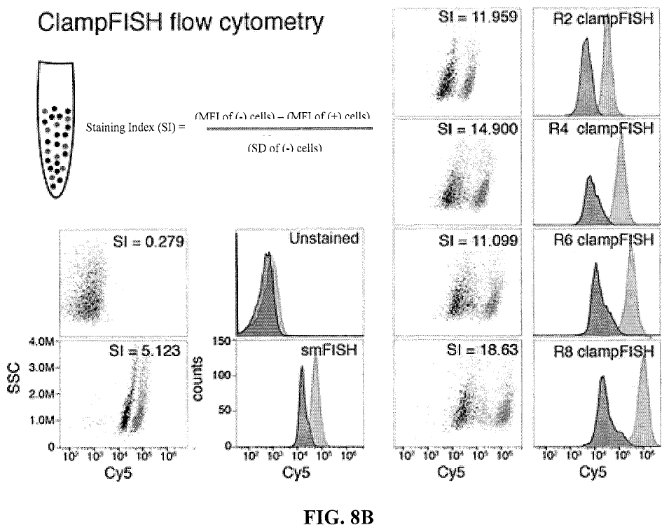

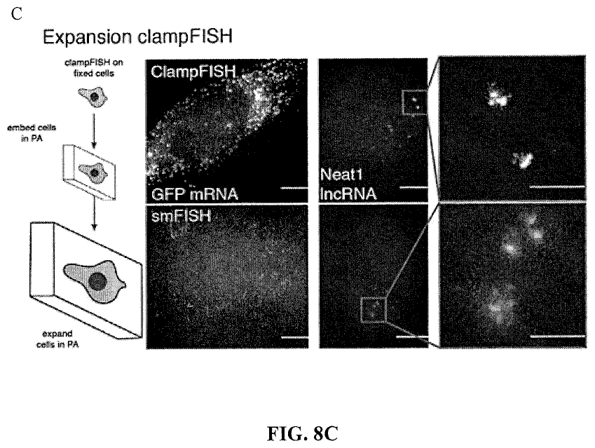

Owing to its low signal intensity, single molecule FISH typically requires using a microscope equipped with a high numerical aperture, high magnification objective, typically requiring oil immersion. For high-throughput screening applications, a low magnification air objective is preferable. ClampFISH has the potential to amplify RNA FISH signals to the point where low magnification microscopy becomes feasible (FIG. 2B). To test this, 20% WM983b cells stably expressing GFP were mixed with 80% WM983b cells and probed for GFP mRNA using clampFISH probes. Using single molecule RNA FISH, individual RNA spots were discerned using the 1.4 NA 60× and 100× objectives, but the spots were dim, not punctate using the 20× objective for smFISH and not visible at all for the 10× objective. Using clampFISH, the positive cells were clearly discernible at both 20× and 10× magnification (FIG. 2B. FIGS. 7A-7B and FIGS. 8A-8C).

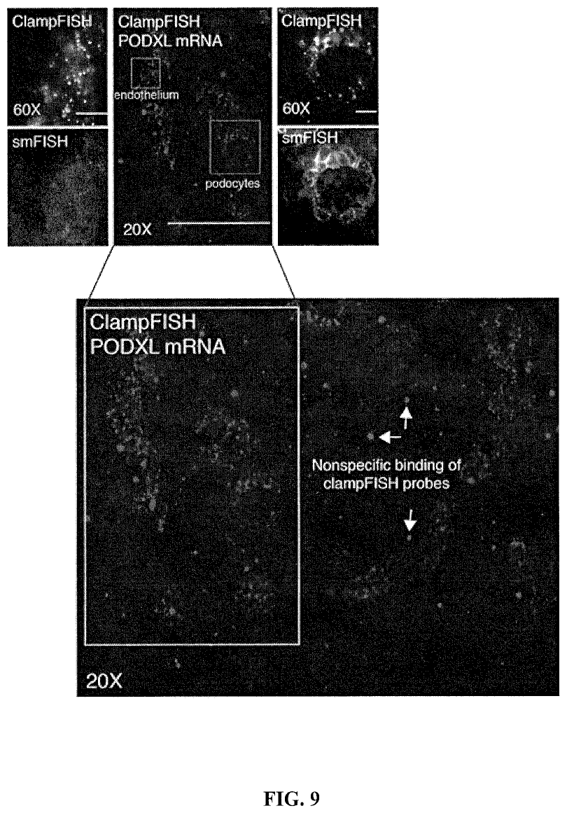

Primary tissue samples typically exhibit high background levels that contribute to a low signal-to-noise ratio using smFISH and therefore require high magnification microscopy to discern positive signal from background. At high magnification, large structural features of the tissue are not easily assessed. To reduce the magnification and increase the visible area while still viewing individual RNAs, 4-rounds of clampFISH were applied to mouse kidney samples and probed for PODXL mRNA, an established mRNA marker that is highly expressed in podocytes, and specific expression of clampFISH signal in the appropriate regions was observed (FIG. 2A and FIG. 9). Interestingly, clampFISH clearly revealed PODXL mRNAs in the kidney endothelium at low magnification that were only faintly visible by smFISH at high magnification. This expression pattern is consistent with previous findings that PODXL is expressed at low levels in the kidney endothelium (Horrillo et al., Eur. J. Cell Biol. 95, 265-276 (2016)).

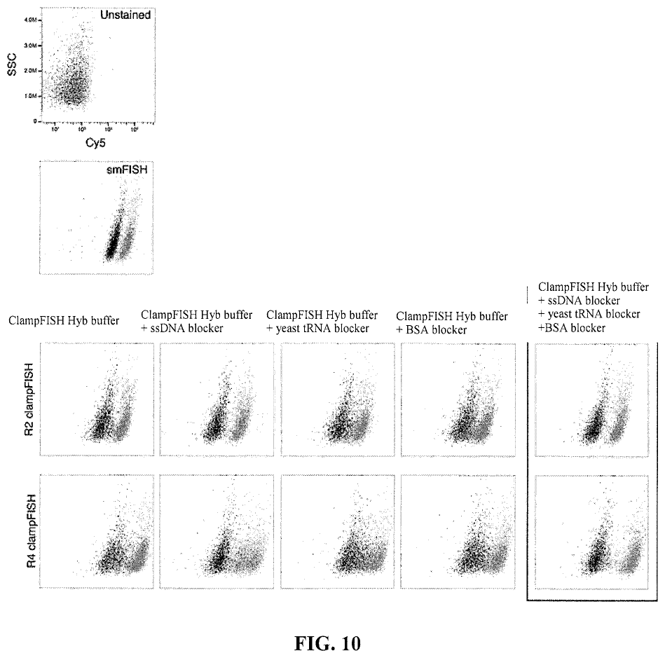

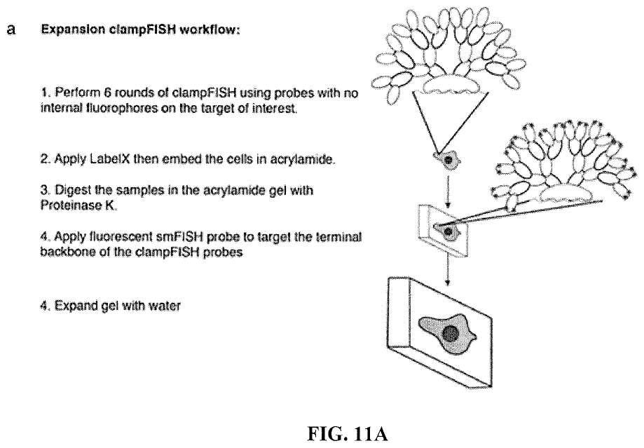

Another important application that clampFISH enables is cell separation by flow cytometry based on RNA expression, an application for which single molecule RNA FISH typically does not produce enough signal (Klemm et al., Nat. Methods 11, 549-551 (2014); Bushkin et al., J. Immunol. 194, 836-841 (2015)). ClampFISH was applied to a mixed population of MDA-MB 231 cells with and without GFP expression and analyzed the cells by flow cytometry (FIG. 2B and FIG. 10), using GFP fluorescence as an independent measure of the specificity of clampFISH signal. A separation of GFP positive cells by clampFISH signal was observed as early as 2 rounds of amplification, and a 1/2 decade shift in fluorescence intensity was observed in the GFP positive population with every 2 rounds of amplification thereafter (FIG. 2B and FIG. 10).

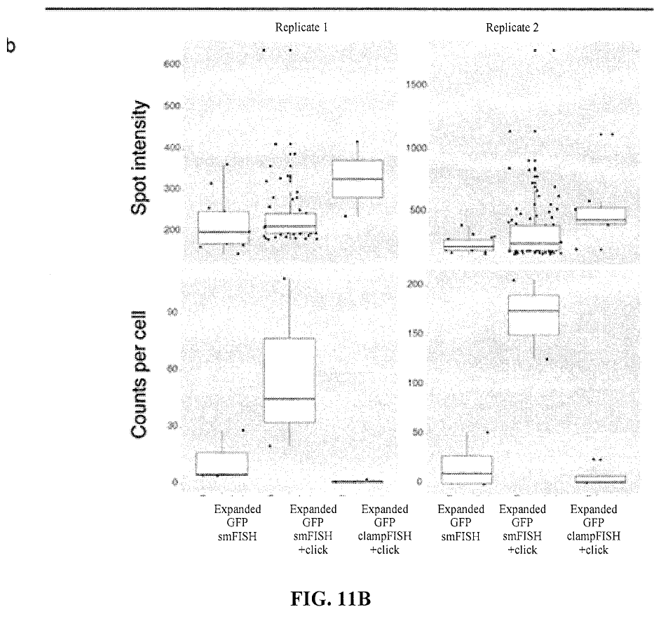

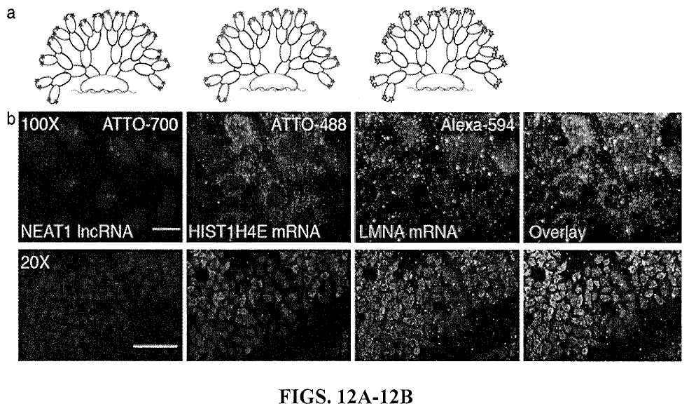

Amplification of RNA signal can also be used as a complementary method for a newly developed expansion microscopy technique that achieves super-resolution microscopy via the physical expansion of cells embedded in polymeric hydrogels (Chen et al., Science 347, 543-548 (2015); Chen et al., Nat. Methods 13, 679-684 (2016)). Expansion microscopy is powerful in combination with single molecule RNA FISH; however, the physical expansion of cells results in reduced signal intensities, at least partially due to probes dissociating under the low salt conditions required to obtain high levels of hydrogel expansion. Based upon the locking property of clampFISH probes that should allow to maintain signal intensity in the face of these expansion conditions, a clampFISH was performed on GFP expressing cells followed by expansion. High signal intensity was observed on all spots when the click reaction was performed, but with little signal when click was not performed (FIG. 2C). ClampFISH was applied to amplify Neat1, a nuclearly retained long non-coding RNA, and a 10-fold higher signal intensity than with smFISH was observed (FIG. 2C and FIGS. 11A-11B), revealing the ring-like structure associated with this RNA's localization pattern.

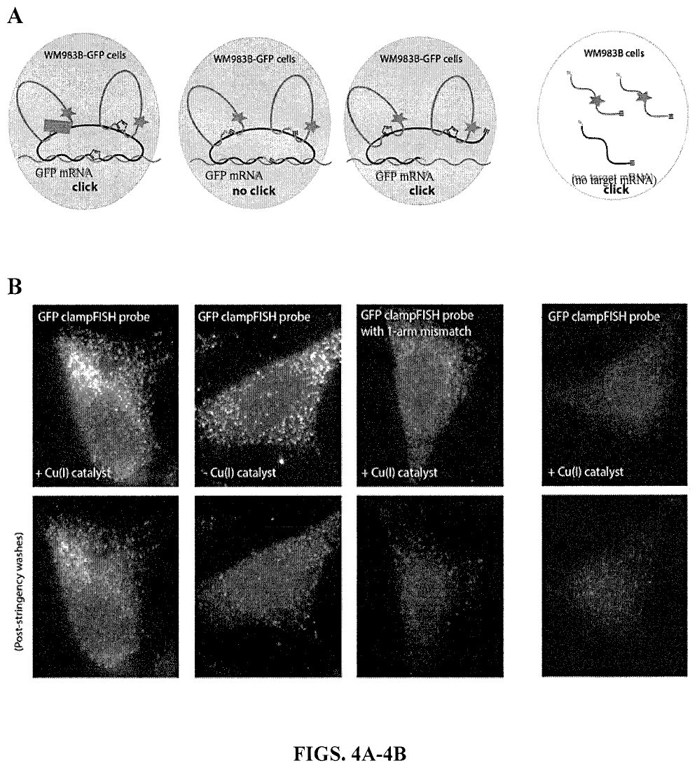

ClampFISH technology can also be useful in a proximity ligation assay. The two hybridizing arms of the clampFISH probes provide an added layer of specificity because the click reaction will not occur unless the azide and the alkyne are in close proximity (FIG. 4). If a one arm is mismatched and does not bind to the target sequence the click reaction will not take place as the azide and alkyne group will not be close enough to click. During the washing steps, a probe with only one arm bound will come off, and this can serve to reduce off-target binding effects. This can also serve as a unique method to assess alternative splicing of a nucleic acid target of interest by targeting splicing junctions, where each binding arm of the primary clampFISH probe targets the nucleotides closest to the respective exons.

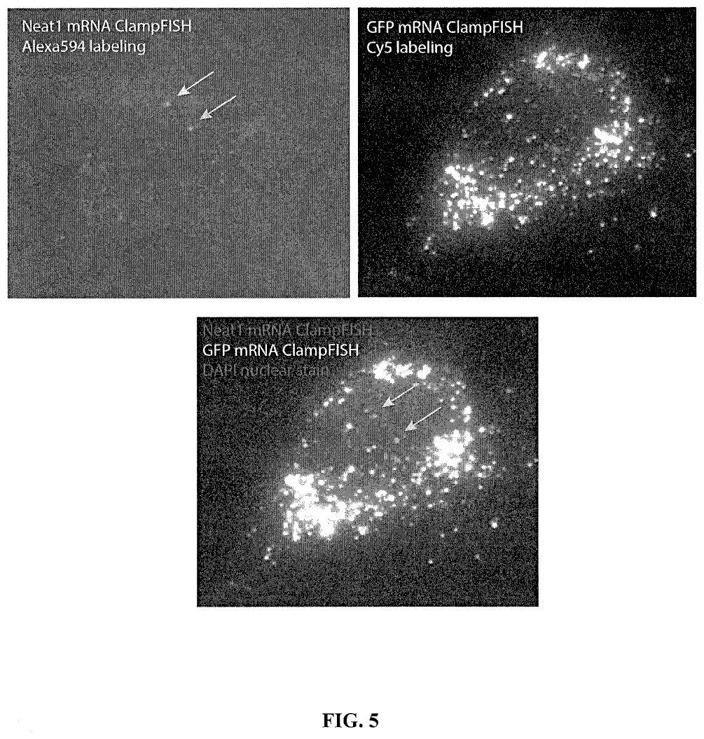

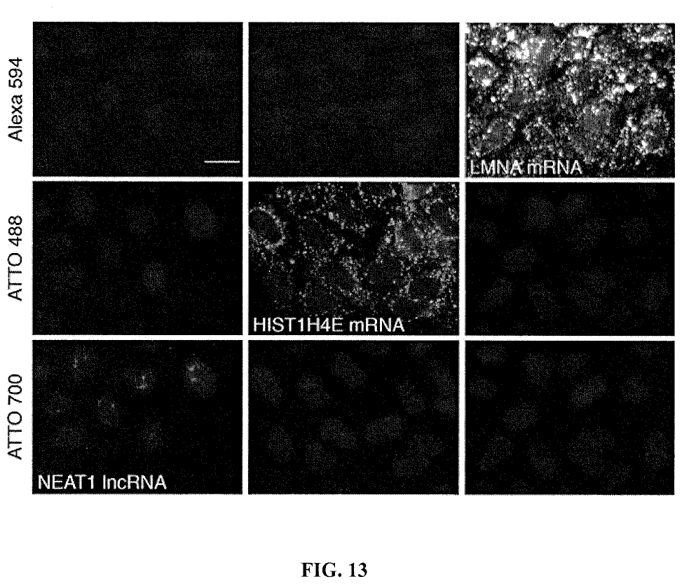

A key design goal for in situ hybridization methods is the ability to detect multiple RNA targets simultaneously. Multiplexing with clampFISH is straightforward in principle because the backbone sequence of the clampFISH probes can easily be changed, allowing one to use multiple independent amplifiers simultaneously. ClampFISH technology can also be useful in a multiplex assay. This process is illustrated in FIG. 5 where NEAT1 clampFISH probes are hybridized to NEAT1 mRNA using a unique backbone sequence. Probes bind specifically to NEAT1 mRNA which is localized to nuclear paraspeckles (arrows). NEAT1 mRNA is detected using an alexa-594 labeled smFISH probe which binds to the terminal and unlabeled NEAT1 clampFISH probe. GFP mRNA is detected using a Cy5 fluorophore built into the backbone sequence of GFP clampFISH probes. As a proof-of-concept, 3 RNA targets were selected, all with distinct expression patterns in HeLa cells: NEAT1, which is found in nuclear paraspeckles of most cells; LMNA, which is found in the cytoplasm of all cells, and HIST1H4E, which expresses only in the subpopulation of cells that are in S phase. These RNAs were amplified with unique sets of non-fluorescent clampFISH probes to 7 rounds, then the terminal backbones were probed with single molecule FISH probes, each labeled with different fluorophores (FIGS. 12A-12B). The signals from the different probe sets were visible, even using low magnification microscopy. A minimal bleedthrough was detected between fluorescence channels (FIG. 13).

### Example 3: Overview

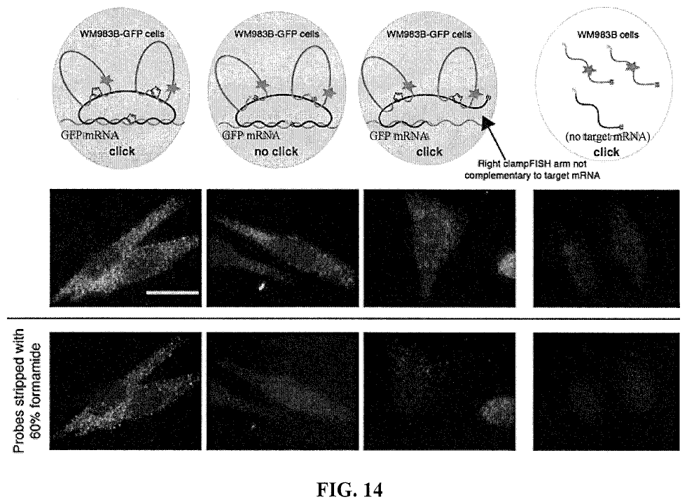

The present invention includes new methods for the fluorescent detection of RNA that combines the specificity of oligonucleotides and a new chemical ligation strategy in order to achieve highly specific and high-gain signal amplification. The resulting clampFISH procedure enables the probing and visualization of individual RNAs on cells and tissues using low powered microscopy and is compatible with expansion microscopy. As described elsewhere herein, by using the methods of this invention, more than 100-fold signal amplification of individual transcripts can be achieved while maintaining minimal off-target binding. The signal intensity obtained using the methods presented herein is so strong that it allows detecting and separating cells based on RNA signal using flow cytometry and low-magnification microscopy, enabling the analysis and separation of large numbers of cells. Interestingly, the combination of probe hybridization and click chemistry moieties on the ends of the primary clampFISH probes behave as a proximity ligation wherein the click reaction will occur only if the two arms are hybridized adjacent to each other (FIG. 14). These data suggest that clampFISH may be useful in specifically probing other difficult-to-image RNA subsets such as splicing junctions, short alternatively spliced variants, or edited RNAs. The methods of this invention exceed nucleic acid based amplification methods known in the art and circumvent enzyme-based amplification schemes generally used in the art and that suffer from poor cell penetration. Particularly, the present chemical ligation step, using a click chemistry reaction, enables stringent wash conditions to reduce background. The results presented herein demonstrate the power of RNA amplification in situ and open up the single-cell and RNA fields for mechanistic studies using high-throughput, analytical methods.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for exponentially amplifying the signal of a fluorescently labeled primary click-amplifying FISH (clampFISH) probe, the method comprising:
(a) hybridizing the primary clampFISH probe to a target nucleic acid in a sample to form a primary sample;
(b) adding a click chemistry agent that covalently locks the primary clampFISH probe to the target nucleic acid in the primary sample;
(c) contacting the primary sample with a set of secondary clampFISH probes that bind to the primary clampFISH probe and adding a click chemistry agent that covalently locks the set of secondary clampFISH probes to the primary clampFISH probe to form a secondary sample;
(d) contacting the secondary sample with a set of tertiary clampFISH probes that bind to each secondary clampFISH probe and adding a click chemistry agent that covalently locks the set tertiary clampFISH probes to each secondary clampFISH probe to form a tertiary sample;
(e) contacting the tertiary sample with a set of secondary clampFISH probes that bind to each tertiary clampFISH probe and adding a click chemistry agent that covalently locks the secondary clampFISH probes to each tertiary clampFISH probe; and,
(f) repeating steps (d) and (e) until a desired level of fluorescent signal is achieved thereby exponentially amplifying the level of fluorescent signal of the primary clampFISH probe.

2. A method for labeling a target nucleic acid in a sample, the method comprising:
(a) contacting the sample with a fixative, thereby producing a fixed sample;
(b) contacting the fixed sample with a hybridization solution, the hybridization solution comprising one or more primary click-amplifying FISH (clampFISH) probes which hybridize to one or more regions of the target nucleic acid and adding a click chemistry agent that covalently locks the one or more primary clampFISH probes to the one or more regions of the target nucleic acid to form a primary sample;
(c) contacting the primary sample with a set of secondary clampFISH probes that bind to the one or more primary clampFISH probes and adding a click chemistry agent that covalently locks the set of secondary clampFISH probes to the one or more primary clampFISH probes to form a secondary sample;
(d) contacting the secondary sample with a set of tertiary clampFISH probes that bind to each secondary clampFISH probe and adding a click chemistry agent that covalently locks the set tertiary clampFISH probes to each secondary clampFISH probe to form a tertiary sample;
(e) contacting the tertiary sample with a set of secondary clampFISH probes that bind to each tertiary clampFISH probes and adding a click chemistry agent that covalently locks the set secondary clampFISH probes to each tertiary clampFISH probe; and,
(f) repeating steps (d) and (e) until a desired level of labeling of the target nucleic acid is achieved.

3. A method for detecting a fluorescently labeled target nucleic acid in a sample, the method comprising:
(a) contacting the sample with a fixative, thereby producing a fixed sample;
(b) contacting the fixed sample with a hybridization solution, the hybridization solution comprising one or more a primary click-amplifying FISH (clampFISH) probes which hybridize to one or more regions of the target nucleic acid and adding a click chemistry agent that covalently locks the one or more primary clampFISH probes to the one or more regions of the target nucleic acid to form a primary sample;
(c) contacting the primary sample with a set of secondary clampFISH probes that bind to the one or more primary clampFISH probes and adding a click chemistry agent that covalently locks the set of secondary clampFISH probes to the one or more primary clampFISH probes to form a secondary sample;
(d) contacting the secondary sample with a set of tertiary clampFISH probes that bind to each secondary clampFISH probe and adding a click chemistry agent that covalently locks the set of tertiary clampFISH probes to each secondary clampFISH probe to form a tertiary sample;
(e) contacting the tertiary sample with a set of secondary clampFISH probes that bind to each tertiary clampFISH probes and adding a click chemistry agent that covalently locks the set secondary clampFISH probes to each tertiary clampFISH probe; and,
(f) repeating steps (d) and (e) until a desired level of fluorescent signal of the labeled target nucleic acid is achieved; and detecting the fluorescent signal of the labeled target nucleic acid wherein the level of fluorescent signal is exponentially amplified.

4. The method of claim 1, wherein step (f) is repeated 1, 2, 3, 4, 5, 6, 7, 8, 9 or 10 times.

5. The method of claim 1, wherein the set of secondary and tertiary clampFISH probes comprises at least 2 probes.

6. The method of claim 5, wherein each probe of the set of secondary and tertiary clampFISH probes binds to a different region of their respective nucleic acid target.

7. The method of claim 1, wherein the length of the primary, secondary and tertiary clampFISH probes is about 150 nucleic acids.

8. The method of claim 1, wherein the primary, secondary and tertiary clampFISH probes are complementary through their 3′ and 5′ ends to two regions of their respective nucleic acid target.

9. The method of claim 1, wherein the 3′ and 5′ ends of each primary, secondary and tertiary clampFISH probe comprise a binding arm and an adapter of about 15 and 10 nucleic acids respectively.

10. The method of claims 1, wherein the 3′ and 5′ ends of each primary, secondary and tertiary clampFISH probe comprise an azide and an alkyne group respectively or an alkyne and an azide group respectively.

11. The method of claim 1, wherein the internal region of each primary, secondary and tertiary clampFISH probe comprises at two separate locations an alkyne and an azide group.

12. The method of claim 1, wherein the click chemistry agent catalyzes an azide-alkyne cycloaddition thereby covalently locking the primary, secondary and tertiary clampFISH probes around the their respective nucleic acid target.

13. The method of claim 1, wherein the click chemistry is catalyzed by copper(I), copper(II) or ruthenium.

14. The method of claim 1, wherein the primary, secondary and tertiary clampFISH probes are labeled by fluorophore.

15. The method of claim 1, where the signal of the labeled primary, secondary and tertiary clampFISH probes is detected by a fluorescent in situ hybridization (FISH).

16. The method of claim 1, wherein the primary, secondary and tertiary clampFISH probes is a DNA.

17. The method of claim 1, wherein the target nucleic acid is a RNA.

18. The method of claim 17, wherein the RNA is selected from the group consisting of messenger RNA, intronic RNA, exonic RNA, and non-coding RNA.

19. The method of claim 1, wherein the target nucleic acid comprises a splice junction.

20. The method of claim 3, wherein the method is used to identify a splice junction in the target nucleic acid.

21. The method of claim 1, wherein the fluorescent signal is amplified more than 10, more than 50, more than 100 and more than 500 folds as compared to a control labeled with a standard FISH or labeled with a primary, secondary and tertiary clampFISH probes but without repeating the step (f).

22. The method of claim 3, wherein the detection of target nucleic acid in a sample is achieved by using at least one method selected from the group consisting of: low-magnification microscopy and flow cytometry.

23. The method of claim 3, wherein the target nucleic acid in a sample is further processed for expansion microscopy.

24. A kit comprising at set of primary, secondary and tertiary click-amplifying FISH (clampFISH) probes and a click chemistry agent for signal amplification and detection of nucleic acids in a sample and instructions for use thereof.


SEQUENCE LISTING (148 sequences)

SEQ ID NO: 1 - DNA, 25 bp, Artificial Sequence
  Synthesized
tgagctagccacttcacatcatagt

SEQ ID NO: 2 - DNA, 25 bp, Artificial Sequence
  clampFISH probe
cccagaggtcaagttacatcatagt

SEQ ID NO: 3 - DNA, 25 bp, Artificial Sequence
  clampFISH probe
ggctcgctcagctatacatcatagt

SEQ ID NO: 4 - DNA, 25 bp, Artificial Sequence
  clampFISH probe
ctgctgcggcctattacatcatagt

SEQ ID NO: 5 - DNA, 25 bp, Artificial Sequence
  clampFISH probe
tcactcctcagaccaacatcatagt

SEQ ID NO: 6 - DNA, 25 bp, Artificial Sequence
  clampFISH probe
aacacccgagcagcgacatcatagt

SEQ ID NO: 7 - DNA, 25 bp, Artificial Sequence
  clampFISH probe
actcacactggccagacatcatagt

SEQ ID NO: 8 - DNA, 25 bp, Artificial Sequence
  clampFISH probe
gaatacagggcttccacatcatagt

SEQ ID NO: 9 - DNA, 25 bp, Artificial Sequence
  clampFISH probe
ctaacgaagtaaagtacatcatagt

SEQ ID NO: 10 - DNA, 25 bp, Artificial Sequence
  clampFISH probe
gatactagataggcaacatcatagt

SEQ ID NO: 11 - DNA, 92 bp, Artificial Sequence
  clampFISH probe
atattcgcagggtatgttagagtgcgagaagtaggtgtcgatcatattaaggggtatgttagagtgcgagaagtaggtgtcgctgagtgttg

SEQ ID NO: 12 - DNA, 92 bp, Artificial Sequence; identical to SEQ ID NO: 11
  clampFISH probe

SEQ ID NO: 13 - DNA, 92 bp, Artificial Sequence; identical to SEQ ID NO: 11
  clampFISH probe

SEQ ID NO: 14 - DNA, 92 bp, Artificial Sequence; identical to SEQ ID NO: 11
  clampFISH probe

SEQ ID NO: 15 - DNA, 92 bp, Artificial Sequence; identical to SEQ ID NO: 11
  clampFISH probe

SEQ ID NO: 16 - DNA, 92 bp, Artificial Sequence; identical to SEQ ID NO: 11
  clampFISH probe

SEQ ID NO: 17 - DNA, 92 bp, Artificial Sequence; identical to SEQ ID NO: 11
  clampFISH probe

SEQ ID NO: 18 - DNA, 92 bp, Artificial Sequence; identical to SEQ ID NO: 11
  clampFISH probe

SEQ ID NO: 19 - DNA, 92 bp, Artificial Sequence; identical to SEQ ID NO: 11
  clampFISH probe

SEQ ID NO: 20 - DNA, 92 bp, Artificial Sequence; identical to SEQ ID NO: 11
  clampFISH probe

SEQ ID NO: 21 - DNA, 25 bp, Artificial Sequence
  clampFISH probe
aagtgactgttgtcccctgaagccc

SEQ ID NO: 22 - DNA, 25 bp, Artificial Sequence
  clampFISH probe
aagtgactgtcaagagcggcgccct

SEQ ID NO: 23 - DNA, 25 bp, Artificial Sequence
  clampFISH probe
aagtgactgtagaccagcgcacccg

SEQ ID NO: 24 - DNA, 25 bp, Artificial Sequence
  clampFISH probe
aagtgactgttgatcatttccaggg

SEQ ID NO: 25 - DNA, 25 bp, Artificial Sequence
  clampFISH probe
aagtgactgtcgccttaactccaca

SEQ ID NO: 26 - DNA, 25 bp, Artificial Sequence
  clampFISH probe
aagtgactgtgtcaatgttgtcccc

SEQ ID NO: 27 - DNA, 25 bp, Artificial Sequence
  clampFISH probe
aagtgactgttcctgcaatgctagg

SEQ ID NO: 28 - DNA, 25 bp, Artificial Sequence
  clampFISH probe
aagtgactgtaaatggtagcctcct

SEQ ID NO: 29 - DNA, 25 bp, Artificial Sequence
  clampFISH probe
aagtgactgtaaagtattatgaaat

SEQ ID NO: 30 - DNA, 25 bp, Artificial Sequence
  clampFISH probe
aagtgactgtagactacaatttgaa

SEQ ID NO: 31 - DNA, 20 bp, Artificial Sequence
  clampFISH probe
ctgcgaatatactatgatgt

SEQ ID NO: 32 - DNA, 20 bp, Artificial Sequence
  clampFISH probe
ctgcgaatatactatgatgt

SEQ ID NO: 33 - DNA, 20 bp, Artificial Sequence
  clampFISH probe
ctgcgaatatactatgatgt

SEQ ID NO: 34 - DNA, 20 bp, Artificial Sequence
  clampFISH probe
ctgcgaatatactatgatgt

SEQ ID NO: 35 - DNA, 20 bp, Artificial Sequence
  clampFISH probe
ctgcgaatatactatgatgt

SEQ ID NO: 36 - DNA, 20 bp, Artificial Sequence
  clampFISH probe
ctgcgaatatactatgatgt

SEQ ID NO: 37 - DNA, 20 bp, Artificial Sequence
  clampFISH probe
ctgcgaatatactatgatgt

SEQ ID NO: 38 - DNA, 20 bp, Artificial Sequence
  clampFISH probe
ctgcgaatatactatgatgt

SEQ ID NO: 39 - DNA, 20 bp, Artificial Sequence
  clampFISH probe
ctgcgaatatactatgatgt

SEQ ID NO: 40 - DNA, 20 bp, Artificial Sequence
  clampFISH probe
ctgcgaatatactatgatgt

SEQ ID NO: 41 - DNA, 20 bp, Artificial Sequence
  clampFISH probe
acagtcacttcaacactcag

SEQ ID NO: 42 - DNA, 20 bp, Artificial Sequence
  clampFISH probe
acagtcacttcaacactcag

SEQ ID NO: 43 - DNA, 20 bp, Artificial Sequence
  clampFISH probe
acagtcacttcaacactcag

SEQ ID NO: 44 - DNA, 20 bp, Artificial Sequence
  clampFISH probe
acagtcacttcaacactcag

SEQ ID NO: 45 - DNA, 20 bp, Artificial Sequence
  clampFISH probe
acagtcacttcaacactcag

SEQ ID NO: 46 - DNA, 20 bp, Artificial Sequence
  clampFISH probe
acagtcacttcaacactcag

SEQ ID NO: 47 - DNA, 20 bp, Artificial Sequence
  clampFISH probe
acagtcacttcaacactcag

SEQ ID NO: 48 - DNA, 20 bp, Artificial Sequence
  clampFISH probe
acagtcacttcaacactcag

SEQ ID NO: 49 - DNA, 20 bp, Artificial Sequence
  clampFISH probe
acagtcacttcaacactcag

SEQ ID NO: 50 - DNA, 20 bp, Artificial Sequence
  clampFISH probe
acagtcacttcaacactcag

SEQ ID NO: 51 - DNA, 25 bp, Artificial Sequence
  clampFISH probe
gcactctaacataccttttaatatt

SEQ ID NO: 52 - DNA, 90 bp, Artificial Sequence
  clampFISH probe
caatgagtcaggagttgtgtttgtggacgaagagcaccatcatattaaggcaggagttgtgtttgtggacgaagagcaccataagaatca

SEQ ID NO: 53 - DNA, 25 bp, Artificial Sequence
  clampFISH probe
aagtgactgtggtggttgttgaagt

SEQ ID NO: 54 - DNA, 20 bp, Artificial Sequence
  clampFISH probe
actcattgaatattaaaaca

SEQ ID NO: 55 - DNA, 20 bp, Artificial Sequence
  clampFISH probe
tgatacttggtgattcttat

SEQ ID NO: 56 - DNA, 25 bp, Artificial Sequence
  clampFISH probe
cacctatccacagtcacatcatagt

SEQ ID NO: 57 - DNA, 92 bp, Artificial Sequence; identical to SEQ ID NO: 11
  clampFISH probe

SEQ ID NO: 58 - DNA, 25 bp, Artificial Sequence
  clampFISH probe
aagtgactgttctacaccttcgaac

SEQ ID NO: 59 - DNA, 20 bp, Artificial Sequence
  clampFISH probe
ctgcgaatatactatgatgt

SEQ ID NO: 60 - DNA, 20 bp, Artificial Sequence
  clampFISH probe
acagtcacttcaacactcag

SEQ ID NO: 61 - DNA, 20 bp, Artificial Sequence
  clampFISH probe
cctacttctcgcactctaac

SEQ ID NO: 62 - DNA, 25 bp, Artificial Sequence
  clampFISH probe
gccgcgaccagacatacatcatagt

SEQ ID NO: 63 - DNA, 25 bp, Artificial Sequence
  clampFISH probe
cgcctcctttacccaacatcatagt

SEQ ID NO: 64 - DNA, 25 bp, Artificial Sequence
  clampFISH probe
ttatctcgcaggaccacatcatagt

SEQ ID NO: 65 - DNA, 25 bp, Artificial Sequence
  clampFISH probe
ccggatggcaggcttacatcatagt

SEQ ID NO: 66 - DNA, 25 bp, Artificial Sequence
  clampFISH probe
tgcgcttgacaccccacatcatagt

SEQ ID NO: 67 - DNA, 25 bp, Artificial Sequence
  clampFISH probe
ccgcgagtctcctcgacatcatagt

SEQ ID NO: 68 - DNA, 90 bp, Artificial Sequence
  clampFISH probe
atattcgcagtgcattactggtagtggtaggtagacattcatagtacagatgcattactggtagtggtaggtagacattcctgagtgttg

SEQ ID NO: 69 - DNA, 90 bp, Artificial Sequence; identical to SEQ ID NO: 68
  clampFISH probe

SEQ ID NO: 70 - DNA, 90 bp, Artificial Sequence; identical to SEQ ID NO: 68
  clampFISH probe

SEQ ID NO: 71 - DNA, 90 bp, Artificial Sequence; identical to SEQ ID NO: 68
  clampFISH probe

SEQ ID NO: 72 - DNA, 90 bp, Artificial Sequence; identical to SEQ ID NO: 68
  clampFISH probe

SEQ ID NO: 73 - DNA, 90 bp, Artificial Sequence; identical to SEQ ID NO: 68
  clampFISH probe

SEQ ID NO: 74 - DNA, 25 bp, Artificial Sequence
  clampFISH probe
aagtgactgttccctttccgccttt

SEQ ID NO: 75 - DNA, 25 bp, Artificial Sequence
  clampFISH probe
aagtgactgttacggtgacgcttag

SEQ ID NO: 76 - DNA, 25 bp, Artificial Sequence
  clampFISH probe
aagtgactgtgtaatgccctggatg

SEQ ID NO: 77 - DNA, 25 bp, Artificial Sequence
  clampFISH probe
aagtgactgtgcgacgagcaaggcg

SEQ ID NO: 78 - DNA, 25 bp, Artificial Sequence
  clampFISH probe
aagtgactgtagatgagaccagaaa

SEQ ID NO: 79 - DNA, 25 bp, Artificial Sequence
  clampFISH probe
aagtgactgtaacaccttcagaacc

SEQ ID NO: 80 - DNA, 20 bp, Artificial Sequence
  clampFISH probe
ctgcgaatatactatgatgt

SEQ ID NO: 81 - DNA, 20 bp, Artificial Sequence
  clampFISH probe
ctgcgaatatactatgatgt

SEQ ID NO: 82 - DNA, 20 bp, Artificial Sequence
  clampFISH probe
ctgcgaatatactatgatgt

SEQ ID NO: 83 - DNA, 20 bp, Artificial Sequence
  clampFISH probe
ctgcgaatatactatgatgt

SEQ ID NO: 84 - DNA, 20 bp, Artificial Sequence
  clampFISH probe
ctgcgaatatactatgatgt

SEQ ID NO: 85 - DNA, 20 bp, Artificial Sequence
  clampFISH probe
ctgcgaatatactatgatgt

SEQ ID NO: 86 - DNA, 20 bp, Artificial Sequence
  clampFISH probe
acagtcacttcaacactcag

SEQ ID NO: 87 - DNA, 20 bp, Artificial Sequence
  clampFISH probe
acagtcacttcaacactcag

SEQ ID NO: 88 - DNA, 20 bp, Artificial Sequence
  clampFISH probe
acagtcacttcaacactcag

SEQ ID NO: 89 - DNA, 20 bp, Artificial Sequence
  clampFISH probe
acagtcacttcaacactcag

SEQ ID NO: 90 - DNA, 20 bp, Artificial Sequence
  clampFISH probe
acagtcacttcaacactcag

SEQ ID NO: 91 - DNA, 20 bp, Artificial Sequence
  clampFISH probe
acagtcacttcaacactcag

SEQ ID NO: 92 - DNA, 90 bp, Artificial Sequence; identical to SEQ ID NO: 52
  clampFISH probe

SEQ ID NO: 93 - DNA, 25 bp, Artificial Sequence
  clampFISH probe
aagtgactgtgaatgtctacctacc

SEQ ID NO: 94 - DNA, 20 bp, Artificial Sequence
  clampFISH probe
actcattgaatattaaaaca

SEQ ID NO: 95 - DNA, 20 bp, Artificial Sequence
  clampFISH probe
tgatacttggtgattcttat

SEQ ID NO: 96 - DNA, 25 bp, Artificial Sequence
  clampFISH probe
caaacacaactcctgacatcatagt

SEQ ID NO: 97 - DNA, 90 bp, Artificial Sequence; identical to SEQ ID NO: 68
  clampFISH probe

SEQ ID NO: 98 - DNA, 25 bp, Artificial Sequence
  clampFISH probe
aagtgactgtggtgctcttcgtcca

SEQ ID NO: 99 - DNA, 20 bp, Artificial Sequence
  clampFISH probe
ctgcgaatatactatgatgt

SEQ ID NO: 100 - DNA, 20 bp, Artificial Sequence
  clampFISH probe
acagtcacttcaacactcag

SEQ ID NO: 101 - DNA, 20 bp, Artificial Sequence
  clampFISH probe
tacctaccactaccagtaat

SEQ ID NO: 102 - DNA, 25 bp, Artificial Sequence
  clampFISH probe
ctgcagcctgttctcacatcatagt

SEQ ID NO: 103 - DNA, 25 bp, Artificial Sequence
  clampFISH probe
gatgttcttctggaaacatcatagt

SEQ ID NO: 104 - DNA, 25 bp, Artificial Sequence
  clampFISH probe
gtcgggtctcatgacacatcatagt

SEQ ID NO: 105 - DNA, 25 bp, Artificial Sequence
  clampFISH probe
cagctgcttctggagacatcatagt

SEQ ID NO: 106 - DNA, 25 bp, Artificial Sequence
  clampFISH probe
gtcctccaggtctcgacatcatagt

SEQ ID NO: 107 - DNA, 25 bp, Artificial Sequence
  clampFISH probe
acttgttgcgcagccacatcatagt

SEQ ID NO: 108 - DNA, 25 bp, Artificial Sequence
  clampFISH probe
gtacgcaggctgttcacatcatagt

SEQ ID NO: 109 - DNA, 92 bp, Artificial Sequence
  clampFISH probe
atattcgcaggaggatgaggttatgcgacttggataggtgatcatattaagggaggatgaggttatgcgacttggataggtgctgagtgttg

SEQ ID NO: 110 - DNA, 92 bp, Artificial Sequence; identical to SEQ ID NO: 109
  clampFISH probe

SEQ ID NO: 111 - DNA, 92 bp, Artificial Sequence; identical to SEQ ID NO: 109
  clampFISH probe

SEQ ID NO: 112 - DNA, 92 bp, Artificial Sequence; identical to SEQ ID NO: 109
  clampFISH probe

SEQ ID NO: 113 - DNA, 92 bp, Artificial Sequence; identical to SEQ ID NO: 109
  clampFISH probe

SEQ ID NO: 114 - DNA, 92 bp, Artificial Sequence; identical to SEQ ID NO: 109
  clampFISH probe

SEQ ID NO: 115 - DNA, 92 bp, Artificial Sequence; identical to SEQ ID NO: 109
  clampFISH probe

SEQ ID NO: 116 - DNA, 25 bp, Artificial Sequence
  clampFISH probe
aagtgactgtttcctccttcatggt

SEQ ID NO: 117 - DNA, 25 bp, Artificial Sequence
  clampFISH probe
aagtgactgtcagctcctcactgta

SEQ ID NO: 118 - DNA, 25 bp, Artificial Sequence
  clampFISH probe
aagtgactgttgtcaatctccacca

SEQ ID NO: 119 - DNA, 25 bp, Artificial Sequence
  clampFISH probe
aagtgactgtcgcctccttggctgc

SEQ ID NO: 120 - DNA, 25 bp, Artificial Sequence
  clampFISH probe
aagtgactgtctcacgggccagtga

SEQ ID NO: 121 - DNA, 25 bp, Artificial Sequence
  clampFISH probe
aagtgactgtactggtcctcattgg

SEQ ID NO: 122 - DNA, 25 bp, Artificial Sequence
  clampFISH probe
aagtgactgtgagttgatgagagcc

SEQ ID NO: 123 - DNA, 20 bp, Artificial Sequence
  clampFISH probe
ctgcgaatatactatgatgt

SEQ ID NO: 124 - DNA, 20 bp, Artificial Sequence
  clampFISH probe
ctgcgaatatactatgatgt

SEQ ID NO: 125 - DNA, 20 bp, Artificial Sequence
  clampFISH probe
ctgcgaatatactatgatgt

SEQ ID NO: 126 - DNA, 20 bp, Artificial Sequence
  clampFISH probe
ctgcgaatatactatgatgt

SEQ ID NO: 127 - DNA, 20 bp, Artificial Sequence
  clampFISH probe
ctgcgaatatactatgatgt

SEQ ID NO: 128 - DNA, 20 bp, Artificial Sequence
  clampFISH probe
ctgcgaatatactatgatgt

SEQ ID NO: 129 - DNA, 20 bp, Artificial Sequence
  clampFISH probe
ctgcgaatatactatgatgt

SEQ ID NO: 130 - DNA, 20 bp, Artificial Sequence
  clampFISH probe
acagtcacttcaacactcag

SEQ ID NO: 131 - DNA, 20 bp, Artificial Sequence
  clampFISH probe
acagtcacttcaacactcag

SEQ ID NO: 132 - DNA, 20 bp, Artificial Sequence
  clampFISH probe
acagtcacttcaacactcag

SEQ ID NO: 133 - DNA, 20 bp, Artificial Sequence
  clampFISH probe
acagtcacttcaacactcag

SEQ ID NO: 134 - DNA, 20 bp, Artificial Sequence
  clampFISH probe
acagtcacttcaacactcag

SEQ ID NO: 135 - DNA, 20 bp, Artificial Sequence
  clampFISH probe
acagtcacttcaacactcag

SEQ ID NO: 136 - DNA, 20 bp, Artificial Sequence
  clampFISH probe
acagtcacttcaacactcag

SEQ ID NO: 137 - DNA, 25 bp, Artificial Sequence
  clampFISH probe
cataacctcatcctcttttaatatt

SEQ ID NO: 138 - DNA, 92 bp, Artificial Sequence
  clampFISH probe
ttcaatgagtagtgcggtggttaagcgagttgtgaaggatatcatattaaggagtgcggtggttaagcgagttgtgaaggatataagaatca

SEQ ID NO: 139 - DNA, 25 bp, Artificial Sequence
  clampFISH probe
aagtgactgtcacctatccaagtcg

SEQ ID NO: 140 - DNA, 20 bp, Artificial Sequence
  clampFISH probe
actcattgaatattaaaaca

SEQ ID NO: 141 - DNA, 20 bp, Artificial Sequence
  clampFISH probe
tgatacttggtgattcttat

SEQ ID NO: 142 - DNA, 25 bp, Artificial Sequence
  clampFISH probe
cttaaccaccgcactacatcatagt

SEQ ID NO: 143 - DNA, 92 bp, Artificial Sequence; identical to SEQ ID NO: 109
  clampFISH probe

SEQ ID NO: 144 - DNA, 25 bp, Artificial Sequence
  clampFISH probe
aagtgactgtatccttcacaactcg

SEQ ID NO: 145 - DNA, 20 bp, Artificial Sequence
  clampFISH probe
ctgcgaatatactatgatgt

SEQ ID NO: 146 - DNA, 20 bp, Artificial Sequence
  clampFISH probe
acagtcacttcaacactcag

SEQ ID NO: 147 - DNA, 20 bp, Artificial Sequence
  clampFISH probe
atccaagtcgcataacctca

SEQ ID NO: 148 - DNA, 25 bp, Artificial Sequence
  clampFISH probe
actaccagtaatgcattttaatatt